(19) United States
(12) Patent Application Publication
(10) Pub. No.: US 2025/0259708 A1
(43) Pub. Date: Aug. 14, 2025

(54) BIOLOGICAL TISSUE SELECTION AND ANALYSIS FOR ASSAY CREATION AND OUTCOME ESTIMATION

(71) Applicant: Magellan Bioanalytics, Inc., Pleasant Grove, UT (US)

(72) Inventors: Dan Reed Olsen, JR., Orem, UT (US); Marc David Hansen, Pleasant Grove, UT (US)

(73) Assignee: Magellan Bioanalytics, Inc., Pleasant Grove, UT (US)

(21) Appl. No.: 19/049,462

(22) Filed: Feb. 10, 2025

Related U.S. Application Data
(60) Provisional application No. 63/551,424, filed on Feb. 8, 2024.

(51) Int. Cl. G16B 40/10; C12Q 1/6869; G01N 33/68

(52) U.S. Cl. CPC G16B 40/10; C12Q 1/6869; G01N 33/6848

(57) ABSTRACT

Systems and methods are provided for identifying salient biological features from molecular data to differentiate between biological outcomes. A biological sample, such as blood, plasma, serum, or liquefied tissue, is obtained from a subject and processed using chemical, mechanical, and/or enzymatic treatment. The prepared sample undergoes mass spectrometry and/or RNA sequencing to generate molecular feature data. A training set of feature vectors is generated. Each feature vector is associated with a known biological outcome. Decision values are computed to determine the features most relevant for outcome differentiation. Features with low decision values may be discarded. A classification model is constructed using the pruned feature set and applied to new biological samples to predict outcomes with a confidence score.

### RELATED APPLICATIONS

This application claims priority to and benefit under 35 U.S.C. § 119 to U.S. Provisional Patent Application No. 63/551,424, filed on Feb. 8, 2024, titled “Biological Tissue Selection and Analysis for Assay Creation and Outcome Estimation,” which application is hereby incorporated by reference in its entirety.

### TECHNICAL FIELD

This disclosure relates to biological tissue sampling and analysis. This disclosure relates to systems and methods for biological tissue sampling, as well as molecular analysis. Specifically, this disclosure relates to techniques for processing biological samples, such as blood, plasma, serum, or liquefied tissue. The disclosure also relates to statistical and machine learning-based systems and methods.

### BRIEF DESCRIPTION OF THE DRAWINGS

FIG. 1 is a schematic graph illustrating an example chromatography process used to separate molecular components of a sample analyte based on differential retention times, according to one embodiment.

FIG. 2 is a three-dimensional representation of combined chromatography and mass spectrometry analysis, according to one embodiment.

FIG. 3 is a structured overview of feature vector construction across multiple individuals and processes, according to one embodiment.

FIG. 4 depicts a flow diagram of a process for creating an assay classifier function, according to one embodiment.

FIG. 5 is a conceptual graph illustrating a voting classifier approach, according to one embodiment.

FIG. 6 is a schematic representation of a therapeutic information service system, according to one embodiment.

FIG. 7 shows a graph comparing a data decision value set and a random decision value set, according to one embodiment.

FIG. 8 provides two example spectrographs illustrating how the size of quantum features in mass spectrometry data can affect resolution and feature generation, according to one embodiment.

FIG. 9 illustrates a quantum rectangle and a surrounding “halo” rectangle in m/z and RT dimensions, according to one embodiment.

FIGS. 10A-10C show example color mappings of sub-quantum regions for a particular feature and example, highlighting how peaks can be more precisely located within a broader quantum feature.

FIG. 11 depicts color mappings of the same sub-quantum region for different outcomes, showing how molecular signals may differ in location or intensity across outcome categories.

FIGS. 12A-12C provide illustrations of slicing through sub-quantum data in both the m/z and RT dimensions, according to one embodiment.

FIGS. 13A-13C show summation graphs derived from selected sub-regions in a sub-quantum data display, according to one embodiment.

FIG. 14 is a grid of color mappings for various features, according to one embodiment.

FIG. 15 is a graph showing three overlapping outcome distributions, according to one embodiment.

FIG. 16 presents a feature-value partition visualization, highlighting the use of a boundary to separate two outcome categories, according to one embodiment.

FIG. 17 is another visualization example illustrating an outcome distribution scenario where no single boundary perfectly separates two outcome groups, according to one embodiment.

FIG. 18 shows a non-binary feature-value partition example with multiple boundaries used to classify more than two outcome categories, according to one embodiment.

FIG. 19 is a graph of a probability distribution and associated sample points, according to one embodiment.

FIG. 20 illustrates an improved model for estimating probability distributions by positioning midpoints between observed sample points, according to one embodiment.

FIG. 21 depicts three probability distributions for a feature across different outcomes where distributions overlap partially, according to one embodiment.

FIG. 22 provides a schematic representation of a decision value computation process for a feature, according to one embodiment.

FIG. 23 compares three features that separate two outcome distributions, according to one embodiment.

FIG. 24 shows additional data related to a feature showing how maximum and minimum sample values and partition points affect outcome classification, according to one embodiment.

FIG. 25 illustrates an example point A used to indicate how a confidence value can be assigned based on the location of a point relative to partition boundaries, according to one embodiment.

FIG. 26 depicts a training set combining feature vectors and outcomes across multiple processes and individuals, according to one embodiment.

FIG. 27 is a chart comparing the number and decision values of features identified by four different processes, according to one embodiment.

FIG. 28 is a parallel-axes plot of a single feature across an ordered series of outcomes showing how the abundance of the feature changes, according to one embodiment.

FIG. 29 is a multi-feature parallel-axes graph plotting multiple features to reveal broader trends across an ordered sequence of outcomes, according to one embodiment.

FIGS. 30A and 30B show interactive selection rectangles on the parallel axes display used to filter or highlight subsets of features that match specific criteria, according to one embodiment.

FIGS. 31A and 31B further demonstrate multiple selection rectangles on a parallel axes chart to de-emphasize or remove features outside the selected regions, according to one embodiment.

FIG. 32 is a decision value display across multiple binary decisions showing how feature importance can vary when comparing different outcome pairs, according to one embodiment.

FIG. 33 shows the decision value display of claim 32 with the additional interactive selection of a subset of features, according to one embodiment.

FIG. 34 is an abundance value display across an ordered outcome series, according to one embodiment.

FIG. 35 shows another abundance value display with a selection rectangle identifying features that spike in a particular outcome category, according to one embodiment.

FIG. 36 is a bi-cluster display, including a heat map of expression values and two cluster trees, according to one embodiment.

FIG. 37 demonstrates cluster selections on the example tree and feature tree, according to one embodiment.

### DETAILED DESCRIPTION

In various embodiments, the systems and methods described herein include taking liquid biological samples and analyzing them to extract molecular information relevant to biological processes to determine or distinguish between different “outcomes.” For example, a blood sample may be processed to determine the disease stage of a breast cancer patient. In such an embodiment, the analysis and test may operate to distinguish between different possible disease stages (e.g., different possible outcomes). The test and analysis may operate to reveal information about the biological processes involved in breast cancer progression. A liquified tissue sample may be analyzed to predict the efficacy of a particular cancer therapy.

Some aspects of the systems and methods described herein include processes to obtain liquid samples from subjects, along with their associated outcome data, to develop diagnostic and/or predictive models for specific outcomes using chromatography, mass-spectrometry, and/or machine learning. The approaches described herein may include analyzing molecular differences between samples corresponding to different outcomes. These approaches enable the identification of biologically significant variations, which can be used to inform disease classification and therapy selection in some instances.

Analyzing biological data presents unique challenges, including the difficulty associated with managing and processing high-dimensional feature spaces and distinguishing relevant molecular markers from background noise. The systems and methods described herein provide solutions to these challenges. As compared to existing approaches and solutions, the presently described systems and methods allow for more precise outcome predictions and improved therapeutic guidance.

As a specific example, blood samples may be collected from a cohort of patients diagnosed with breast cancer (e.g., one hundred patients). Each patient's disease stage may be determined through existing clinical tests. The existing clinical tests are usually expensive and often invasive. In addition to the blood samples, patient data, such as demographic and medical information, may be collected for each patient. Examples of such patient data include but are not limited to, age, locale (general geographic region or specific location), race, current medication, lab results, and/or disease stage. Each blood sample may be analyzed using mass-spectrometry to generate a large number of digital values or “molecular features.” The molecular features may be used alone or combined (e.g., augmented by) various patient data to form a “feature vector.” In this specific example, the stage of the breast cancer of each respective patient is an “outcome.” A training set of feature vector/outcome pairs maps molecular profiles to disease stages.

Breast cancer is merely used as an example in the above description. More generally, a training set of feature vectors and corresponding outcomes maps molecular profiles to outcomes. It is appreciated that there are a wide number of therapy decisions and information to which the techniques described herein may be applied.

A feature vector, as used herein, is a one-dimensional array of numerical values that represent biological and, in some instances, demographic information (or other patient-specific data) associated with a specific biological tissue type. A feature vector is generated by obtaining, preparing, and analyzing tissue samples to produce a numerical representation of molecular and/or biological characteristics.

An outcome is any indication that separates one biological state from another. Some outcome sets are binary. An outcome set includes two or more outcomes for comparison or distinguishing therebetween. Examples of binary outcome sets include pneumonia/not pneumonia, anemia/not anemia, and early-stage cancer/late-stage cancer. An example of a non-binary outcome set related to cancer staging may include Stage I, Stage II, Stage III, and Stage IV. By combining an outcome with a feature vector, evidence can be acquired of what biology characterizes that particular outcome. An outcome can be combined with a feature vector. An outcome combined with a feature vector provides evidence of biological patterns associated with that outcome. The combination of an outcome and a feature vector can be used as a training example, and multiple training examples may be aggregated into a training set. Training examples within a training set may be associated with the same processes and outcome definitions to allow for consistent comparisons across different samples.

Some of the infrastructure that can be used with embodiments disclosed herein is already available, such as general-purpose computers, computer programming tools and techniques, digital storage media, and communication links. Many of the systems, subsystems, modules, components, and the like that are described herein may be implemented as hardware, firmware, and/or software. Various systems, subsystems, modules, and components are described in terms of the function(s) they perform because such a wide variety of possible implementations exist. For example, it is appreciated that many existing programming languages, hardware devices, frequency bands, circuits, software platforms, networking infrastructures, and/or data stores may be utilized alone or in combination to implement a specific control function.

It is also appreciated that two or more of the elements, devices, systems, subsystems, components, modules, etc., that are described herein may be combined as a single element, device, system, subsystem, module, or component. Moreover, many of the elements, devices, systems, subsystems, components, and modules may be duplicated or further divided into discrete elements, devices, systems, subsystems, components, or modules to perform subtasks of those described herein. Any aspect of any embodiment described herein may be combined with any other aspect of any other embodiment described herein or in the other disclosures incorporated by reference, including all permutations and combinations thereof, consistent with the understanding of one of skill in the art reading this disclosure in the context of such other disclosures.

To the extent used herein, a computing device, system, subsystem, module, driver, or controller may include a processor, such as a microprocessor, a microcontroller, logic circuitry, or the like to implement or execute instructions stored in a non-transitory medium. A processor may include one or more special-purpose processing devices, such as application-specific integrated circuits (ASICs), programmable array logic (PAL), programmable logic array (PLA), a programmable logic device (PLD), field-programmable gate array (FPGA), or another customizable and/or programmable device. The computing device may also include a machine-readable storage device, such as non-volatile memory, static RAM, dynamic RAM, ROM, magnetic memory, optical memory, flash memory, or another transitory or non-transitory computer-readable medium or machine-readable storage media. Various aspects of some of the embodiments described herein may be implemented or enhanced using hardware, software, firmware, or a combination thereof.

The components of some of the disclosed embodiments are described and illustrated in the figures herein to provide specific examples. Many portions thereof could be arranged and designed in a wide variety of different configurations. Furthermore, the features, structures, and operations associated with one embodiment may be applied to or combined with the features, structures, or operations described in conjunction with another embodiment. In many instances, well-known structures, materials, or operations are not shown or described in detail to avoid obscuring aspects of this disclosure. The right to add any described embodiment or feature to any one of the figures and/or as a new figure is explicitly reserved.

The embodiments of the systems and methods provided within this disclosure are not intended to limit the scope of the disclosure but are merely representative of possible embodiments. In addition, the steps of a method do not necessarily need to be executed in any specific order or even sequentially, nor do the steps need to be executed only once, except as explicitly stated or as contextually understood by one of skill in the art.

The presently described systems and methods can be used to identify salient biological features from molecular data to differentiate between biological outcomes. As described below in greater detail, the method may generally include obtaining biological samples, preparing the biological samples to form sample analytes, analyzing the analytes, building feature vectors, labeling or associated known outcomes with the feature vectors, computing decision values for the features, pruning the feature vectors to include only the most salient feature vectors relative to the outcomes, training a classification model (e.g., a machine learning model), and using the trained classification model to predict an outcome for a newly acquired biological sample.

In one example embodiment, a biological sample (e.g., blood, serum, or liquefied tissue) is obtained from a cohort of subjects. Each sample undergoes a sample preparation step to remove or fragment components. The sample preparation may, for example, be used to produce an analyte suitable for mass spectrometry. The analyte is injected into a mass spectrometer equipped with a chromatography column, separating molecular species by retention time (RT) and mass-to-charge ratio (m/z). A computing system parses the resulting mass spectrometry data, quantizes it into molecular feature vectors, and labels each feature vector with a known biological outcome (such as disease vs. no disease or therapy success vs. failure). A decision value is computed for each distinct feature by evaluating its ability to separate outcome classes. Features with low decision values below a reliability threshold (e.g., determined by comparing against “random” decision values) are discarded, forming a pruned, salient feature set. The system then constructs or trains a machine-learning classifier, such as a neural network or decision tree, using the pruned, salient feature set. Once trained, the classification model is stored for future application to new biological samples to generate predicted biological outcomes.

In another example embodiment, the system uses RNA sequencing rather than mass spectrometry. Each sample is subjected to sample preparation that isolates nucleic acids. A sequencing device produces short-read sequences. The system extracts K-mer features from the reads to represent gene expression signatures. The system labels each sample with a known outcome (e.g., cancer stage) and computes a decision value for each K-mer feature across all training samples. A reliability threshold is used to prune low-value features (e.g., those that don't distinguish between outcomes) to reduce the feature space. The resulting set of high-decision-value features (e.g., the pruned, salient feature set) is used to train a model. The model can be applied to new sequencing data to predict outcomes such as a tumor subtype or therapy response.

In yet another embodiment, the system integrates both mass spectrometry and RNA-sequencing data by concatenating the feature vectors derived from both processes. A decision-value calculation is performed on the combined data. Features from both sources that exceed the reliability threshold are retained. The combined model reveals composite biomarkers that, in some instances, are more discriminative than single-technology approaches.

Still, other variations will be apparent to those skilled in the art upon reading this disclosure. For example, the molecular analysis step may employ different chromatographic techniques (e.g., reverse-phase vs. hydrophilic interaction) or multiple parallel columns to enhance coverage of diverse molecular species. Sequencing protocols may target DNA, total RNA, mRNA, or microRNA, depending on the biological question. Feature selection can leverage different statistical or machine-learning metrics to compute decision values, such as GINI impurity, parametric tests, or equiprobable distributions. The pruned feature set may be further clustered or refined via correlation-based approaches to produce “super features” representing entire pathways or molecular complexes. All such variations and their combinations may be adapted to specific diagnostic, prognostic, or therapeutic needs.

### Sample Preparation

Biological samples, such as fluid samples or tissue samples, are obtained from a patient. The process to generate a feature vector from a biological sample may include sample preparation and/or pre-processing. For example, blood samples may be converted into serum by removing coagulated materials from the blood or into plasma by removing cells from the blood. The serum or plasma may be treated using acetonitrile or other reagents to precipitate large, abundant molecules (e.g., super-abundant molecules). This is just one example of a possible preparation. Other examples of biological samples include blood, lymph, urine, spinal fluid, liquefied tissue samples, and/or any of a variety of other bodily sources or liquified tissue samples. These fluids may be subjected to various chemical, mechanical, enzymatic, and/or other treatments to extract a desired sample fluid (i.e., to generate an analyte). The sample preparation technique(s) used is, in many instances, independent of the analytical procedures used to process the prepared sample fluid (analyte).

In some embodiments, all sample fluids used in a training set may use the same preparation techniques. Similarly, any future sample fluids submitted to a subsequent digital assay analysis may also be prepared using the same sample techniques to ensure comparability. Although humans are used as examples throughout this discussion, these same techniques can be applied to many other species.

### Chromatography

In some embodiments, chromatography is used to convert a sample fluid into digital data. In some embodiments, as described below, chromatography is not utilized. Chromatography may, for example, including passing a sample fluid through a chromatography column, which retains various types of molecules under different solution mixtures, allowing differential retention of molecules when a gradient of solution mixtures is passed through the column. A gradient of solution mixtures may then be applied to facilitate the differential retention and elution of molecules, effectively separating them over time. There are a wide number of chromatography columns and techniques, many of which can be used in the context of the presently described systems and methods. In many embodiments, any of a wide variety of chromatography techniques are used to separate different types of molecules in complex mixtures across chromatography run time.

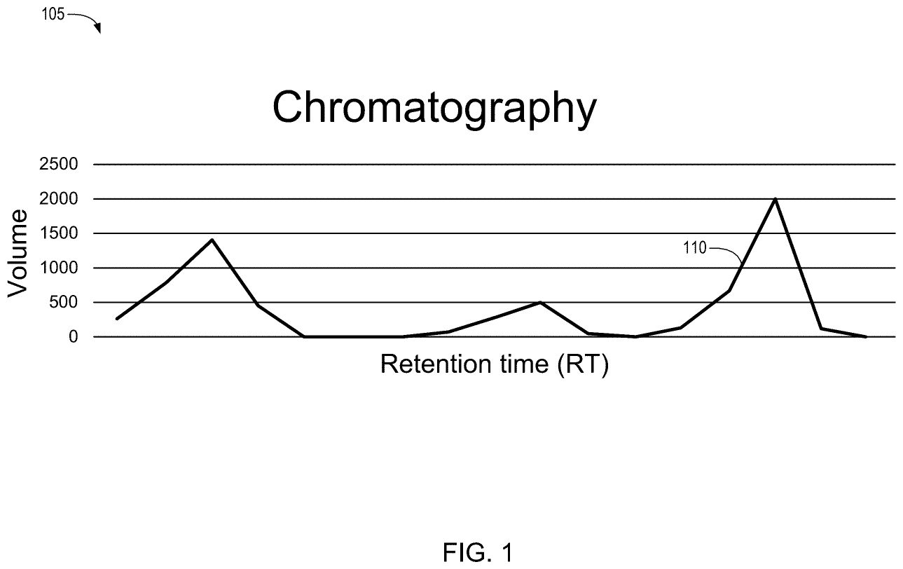

FIG. 1 illustrates a graph 105 of a chromatography process used to separate molecules based on their retention time (RT). Chromatography is one step in converting a sample fluid into digital data. As shown in FIG. 1, molecules elute from the chromatography column at different retention times. The volume of molecules (vertical axis) at different retention times (horizontal axis) is represented by the line 110 of the graph 105. The peaks in line 110 correspond to distinct molecular components in the sample.

Various kinds of molecules will leave the chromatography columns at different times. The time at which a particular molecule leaves the column is its elution time or retention time. In this description, the term retention time (RT) is utilized, though it is interchangeable with elution time. At a particular retention time, there is an abundance, or volume, of molecules that appear. The systems and methods described herein can be used to separate and detect molecular differences rather than to identify particular molecule types. Retention time (RT) can be used as one dimension in a sample's feature data.

One example of chromatography is reverse-phase chromatography, which differentially retains molecules with greater hydrophobicity from those that are more hydrophilic. Hydrophilic interaction chromatography is used to separate polar hydrophilic molecules from non-polar hydrophobic molecules, spreading them out across chromatography run time according to their chemical properties (e.g., how hydrophobic or hydrophilic each molecule is). The presently described systems and methods may utilize one or more different chromatography techniques to distinguish between different chemical properties.

### Mass Spectrometry (MS1)

As previously described, chromatography can be used to separate molecular species. In various embodiments, additional structural and compositional information is obtained by subjecting the molecules to mass spectrometry (MS1). Some embodiments of the presently described systems and methods include directing molecules into a mass spectrometer as they are eluted at a given retention time during chromatography. These molecules are bombarded with electrons to cause molecular fragmentation. Some fragments are negatively charged and/or electrons are added to create negatively charged ions. The charged molecules are accelerated into a high-velocity stream and pass through a magnetic or electrostatic field for analysis.

Charged molecules react to a magnetic field depending on their mass/charge ratio (m/z). The force applied to a molecule depends upon the magnetic field and the charge on the molecule. Given the same mass, a molecule with two extra electrons will have twice the force applied as a molecule charged with only one additional electron. The acceleration of a molecule thus depends on its mass/charge. A molecule of mass m and charge −1 is accelerated the same as a molecule of mass 2m and charge −2.

The acceleration of molecules is measured in a variety of ways, such as time-of-flight, quadrupole filters, ion traps, orbitraps, and others. Mass spectrometers measure the volume or abundance of molecules that have a particular m/z (mass/charge) at a particular point in time. Mass spectrometry techniques may vary in their m/z precision but generally operate to generate an m/z to volume spectrum of the molecules entering the device at a particular time.

When chromatography is used in conjunction with spectrometry (e.g., chromatography before mass spectrometry), the chromatography separates molecules based on their retention time (RT). At each specific retention time or retention time interval, mass spectrometry further differentiates the molecules based on their m/z values and measures their abundance.

Accordingly, as described herein, mass spectrometry analysis may include using a chromatography column to separate molecular components according to differential retention times. The molecular components are then ionized, and intensity signals (e.g., signals indicating the abundance or “abanduance signals”) are detected for a range of mass-to-charge values (M/Z) over a series of retention times. The intensity signals from the mass spectrometer are converted into feature vectors representing molecular abundance.

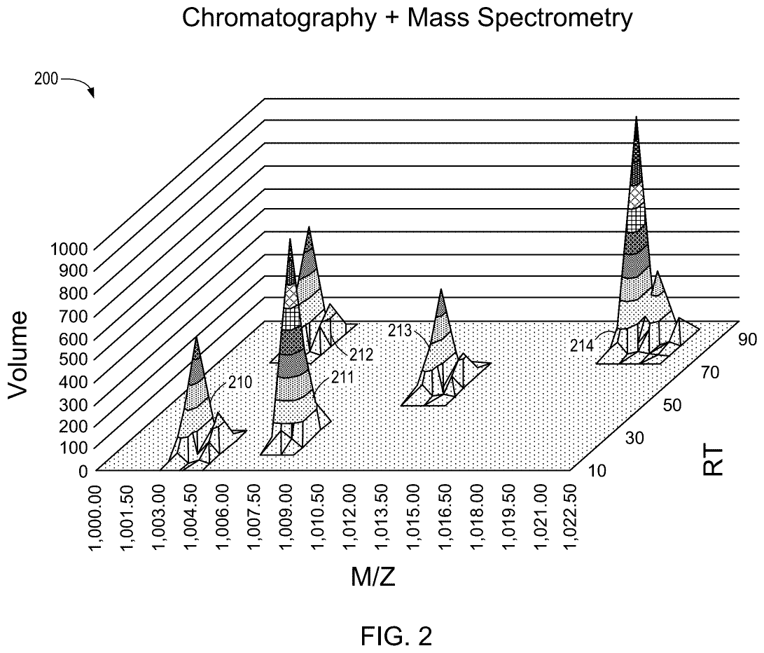

FIG. 2 illustrates a graph 200 with a three-dimensional representation of a combined chromatography and mass spectrometry analysis, where molecular components are separated based on both retention time (RT) and mass-to-charge ratio (m/z), according to one embodiment. The graph 200 plots volume (abundance) along the vertical axis, retention time (RT) along the horizontal axis, and mass-to-charge ratio (m/z) along the depth axis. Peaks 210, 211, 212, 213, and 214 correspond to distinct molecular species detected at specific retention times and m/z values. The combination or integration of chromatography and mass spectrometry enables the identification and quantification of biomolecules within a biological sample. The resulting molecular profile may be used for digital assays and biological outcome predictions.

In various embodiments, the information is digitally encoded as a series of data points, for example, in the form of [m/z, rt, vol] or [mz, rt, vol]. In embodiments in which chromatography is not used, the retention time (RT) rt can be set to zero (0) or omitted.

### Comparable Inputs

The data obtained from the mass spectrometry process is influenced by several factors, including the type of bodily fluid or tissue sampled, the chemical or mechanical techniques applied to convert the sample into a fluid suitable for analysis, the chromatography column and its operational settings, and/or the specific type and precision of the mass spectrometer used. Variations in any of these factors can affect the consistency and comparability of the resulting data. Maintaining uniformity in these conditions within a training set and subsequent assay analysis can help ensure that molecular feature comparisons remain accurate and meaningful. In some embodiments, identical processes and devices may be utilized. In other embodiments, functionally equivalent processes and devices may be utilized without loss of accuracy or functionality, such that the data generated from the mass spectrometry and/or chromatography processes can be reliably used in the analytical techniques described herein.

### Mass-Spec Quantization

The presently described analysis techniques include transforming the [m/z, rt, volume] data produced by a mass spectrometer into feature vectors suitable for additional processing. The raw data (e.g., sample data) from a mass spectrometer can be extensive (e.g., large data sets). For example, a mass spectrometry file (MZML) from a single blood sample may be 1.5 gigabytes in size. Training a machine learning algorithm using 1,000 samples may include over 1.5 terabytes of data. Alternative encoding formats may also be used without affecting the general applicability of the techniques described herein.

To manage the data complexity and size, the dataset to be considered may be reduced and/or normalized into regular features (e.g., structured features), which can be assembled into a feature vector. One approach to reducing the data complexity and size is quantization, which simplifies continuous data by partitioning it into discrete buckets. A quantum feature may be defined as a rectangular region in [m/z, rt] space. The boundaries of each quantum feature may be defined as [minMZ, maxMZ, minRT, maxRT]. Mathematically, a data point [mz, rt] falls into a quantum feature if mz minMZ, mz<maxMZ, rt minRT, and rt<maxRT.

The inclusion or exclusion of boundary values can be adjusted for each comparison without affecting the underlying processing or functionality. A quantum feature space is defined as a list or collection of quantum features that correspond to a structured representation of the mass spectrometry dataset.

There may be more than one measurement triple [m/z, rt, vol] in a mass spectrometry sample that falls within a given [m/z, rt] quantum feature. In such a case, all such measurements may be accumulated by summing them together to create the volume of the quantum feature. Quantum feature boundaries can be created in a variety of ways. A quantum size can be defined along each of the m/z and rt dimensions, and the axis can be divided into a corresponding number of uniform buckets. For example, if the m/z measurements range from 0 to 2,000 and an m/z quantum size of 0.5 is selected, there will be 4,000 quanta along the m/z axis (i.e., 4,000 quantized regions along the m/z axis). Similarly, if the retention time (RT) spans 0 to 3600 seconds with an rt quantum size of 10 seconds (e.g., 10-second intervals), there will be 360 quanta along the rt axis (e.g., 360 quantized regions along the rt axis. This quantization technique results in potentially 4,000×360=1,440,000 quantum features for a given sample. In addition to uniform quantization, alternative strategies such as histogram equalization or adaptive boundary placement may be used to optimize feature resolution and distribution. In other embodiments, the data may be analyzed to place quantum boundaries where there are zeros or local minima in the volume data.

Overlapping quantum features may be defined to better address quantization boundary issues (boundary artifacts). For example, rather than strictly defining two adjacent quantum features as a quantum feature pair [356.0-356.1, 340-350] and [356.0-356.1, 350-360], a third overlapping quantum feature may be included, such that there are three quantum features [356.0-356.1, 340-350], [356.0-356.1, 345-355], and [356.0-356.1, 350-360]. A molecule with a retention time within 345-355 will be accurately captured by the overlapping quantum feature rather than being arbitrarily split. While this approach increases the total number of quanta, it improves the accuracy of molecular representation and minimizes errors introduced by boundary effects. The quantum feature space may be structured as a one-dimensional or two-dimensional vector of features.

Creating large quantum features (large ranges of m/z or rt) can create fewer features and thus take much less space and time for computation. However, large quantum features may lose resolution as many measurements get summed together, and the independent information of each is lost. Small quantum features (small ranges of m/z or rt) can create many more features, take much more space to store, and require more computational effort to handle. The retention of information is high, but the computational cost may become excessive. Also, with small quanta, the peaks in the measurements that correspond to particular molecules may be segmented into separate quanta and lose their biochemical meaning. Problems of over-quantization and possible solutions and approaches for handling over-quantization are described herein.

The result of the quantization step includes a quantum feature space with a list of quantum feature definitions. The quantum feature space may be utilized to produce a feature vector from [m/z, rt, vol] measurements for any given mass spectrometry sample. In some instances, a quantum feature space may be very sparse, meaning many quantum features contain a negligible volume.

In various embodiments, a quantization function is expressible as:

featureIdx=quantize(mz,rt)

The quantize function, quantize(mz,rt), is a computational function or algorithm that maps [m/z and rt] values to a unique feature index. The function may leverage predefined quantum feature boundaries or alternative computational mechanisms to structure the dataset. The presently described systems and methods provide a structured approach for converting mass spectrometry data into feature vectors.

The size of the feature set can be further reduced or refined by removing quantum features that have a total volume below a threshold across all samples. By eliminating quantum features that contain insignificant molecular data, computational storage and processing requirements can be substantially reduced. This optimization approach ensures that the retained features correspond to biologically significant molecular components if the quantization resolution is high enough.

For a given mass spectrometry sample, a feature vector that has its own quantum feature space is obtained, which can be combined with an outcome to produce a training example. When multiple training examples are combined into a training set, the set union of the quantum feature spaces from each example can be computed to form the quantum feature space for the entire training set. If a given training example does not have a particular feature found in the training set's feature space, then its volume is set to zero.

The parsing step is the process of converting the output from a mass spectrometer into a feature space based on a quantum feature space. A human analyst with a human-computer interface may specify the quantization of m/z and rt as well as any desired minimum volume. Quantization is frequently specified as the quantum size for m/z and for rt. From these quantum sizes, a set of candidate quantum features can be generated, and the various mass spectrometer inputs can be parsed.

### Refined DNA/RNA Sequence Features

Similar to the mass spectrometry analysis, a biological sample (e.g., a sample fluid) can be alternatively or additionally analyzed for RNA expression. A sample fluid can be submitted to a DNA/RNA sequencer to identify sequences of DNA or RNA (e.g., after conversion of RNA sequences into corresponding DNA sequences). Much work has been done to sequence the DNA of whole genomes. Whole genome sequences are mostly static in an organism after conception and may have little or no information about health status after that time, particularly when environmental forces impact health status. However, RNA is an engine for protein synthesis, and its expression varies according to the processes that are going on in the organism. When more of particular proteins are desired by the organism, more of the RNA that encode those proteins is produced. RNA also contains information about how the DNA is being transcribed for some purpose. A given sequence of DNA may possibly be transcribed in several ways, producing different RNA sequences. Different RNA sequences produce different proteins. In addition to RNA sequences that encode proteins, cells also convert DNA sequences into RNA products that drive cellular activities directly, either as components of cellular machines or by interacting with DNA or RNA sequences to alter their accessibility. Thus, unlike whole-genome sequencing, which captures an organism's static genetic code, RNA sequencing reflects dynamic gene expression, providing insight into active biological processes.

Both DNA and RNA are sequences of the nucleotide bases adenine(A), guanine(G), cytosine(C), and either thymine(T) for DNA or uracil(U) for RNA. A fragment of DNA or RNA can be characterized by a sequence of these four bases. When a sample fluid is analyzed by a DNA/RNA sequencer, a dataset of DNA/RNA reads is produced. A DNA/RNA read is a short sequence of the four bases, generally a sequence of 50 to 200 bases. In the sequencing process, many such reads are generated. There are usually multiple millions of reads. Results from a DNA/RNA sequencer are generally stored in FASTA or FASTQ file formats, where each read is a sequence of the letters A, G, C, and T or U. Other encodings are possible. In this work, the DNA/RNA information of each sample is encoded as many (millions) of reads, where each read is a sequence of bases.

The systems and methods described herein include techniques based on numeric vectors, so the raw DNA/RNA reads may be converted into structured data using bioinformatics techniques. For example, to develop a feature vector out of DNA/RNA reads, bioinformatics may be used to construct K-mers. For a given analysis, an integer value for K is selected. A K-mer for K=5 would be a sequence of 5 nucleotide bases. For a given read, the system can generate its K-mers by starting at the first base and then counting off K bases. Counting begins again at the next base and generates another K-mer. This process continues until the system moves across the entire read and identifies all the K-mers in that read. For example, given K=6, the RNA read “AGCUUCGUCAAG” translates into the following K-mers: AGCUUC, GCUUCG, CUUCGU, UUCGUC, UCGUCA, CGUCAA, GUCAAG. With millions of reads, it is highly likely that a given K-mer will occur many times in the sample dataset.

A feature space for RNA sequencing can be generated by defining each unique K-mer as a feature. A feature vector for a given sample can be generated by counting the number of times each K-mer is found in the dataset from the sample. This provides an abundance or volume for each K-mer, similar to how molecular abundance is obtained for each quantum feature using mass-spectrometry.

Smaller values of K result in fewer features, but each feature contains less genetic information, and the abundances are higher. Abundance will be driven by an accumulation of counts from identical K-mers found in disparate longer sequences. Larger values of K will generate K-mers that are less common, but each has more genetic information. The likelihood of unique K-mers associated with specific gene sequences is increased but may generate such large numbers of features that computational analysis becomes difficult. In various embodiments, the presently described systems and methods leverage findings that K values between 20 and 30 work well in generating a feature vector that maximizes genetic information while maintaining computational tractability from the reads in a DNA/RNA sequence dataset. In various embodiments, an analyst may use a human-computer interface to specify the value of K to be used.

### Decision Value Pruning

The data generated from mass spectrometry quantization and/or K-mer processing typically contains many more features than samples. While a dataset may include thousands of patient samples (or fewer), the number of extracted molecular features may be in the hundreds or even millions. Many machine learning algorithms function better when the number of samples exceeds the number of features. In various embodiments, the number of features is reduced by pruning features that do not contribute to a target outcome decision. This use of outcomes to decide which features to consider for a particular problem yields information about the biology surrounding the selected outcomes.

Biological processes in an organism are generally connected to a small subset of the many molecules present in the sample. Most of the molecules are unrelated to the biological process being analyzed and/or contribute no meaningful information to the outcome to be detected. A common approach is to study the biochemistry of a process of interest and then search for molecules that relate to that biochemistry.

The approach utilized in the presently described systems and methods is different. The presently described systems and methods analyze all the molecules in the mass spectrometry or RNA-sequenced dataset and consider only those that help separate or even strongly differentiate between target outcomes. Thus, the presently described systems and methods consider and account for molecules that are currently uncharacterized, as well as processes whose biochemistry are not yet understood.

### Input Data

In various embodiments, the data received for analysis may include a one-dimensional array of feature values, where each feature corresponds to a distinct molecular characteristic derived from mass spectrometry quantization or K-mer processing. Each data instance or example may be associated with an outcome value that represents a biological classification or condition. Conceptually, this data can be structured as three distinct arrays, expressible as:

$$\begin{matrix}featureSpace [ featureIdx ] = > [ minMZ , maxMZ , minRT , maxRT ] or K mer \\ features [ example , featureIdx ] = > volume \\ outcomes [ examples ] = > outcome\end{matrix}$$

The three data structures above collectively contain the information used for computing decision values and training predictive models. The specific implementation of these structures may vary, provided that the following conditions are met:
(i) for a given feature index (featureIdx), the bounds of the quantum feature or K-mer can be retrieved,
(ii) for a given example-featureIdx pair, the mass spectrometry or K-mer volume or abundance can be obtained, and
(iii) for a given example, the associated biological outcome can be obtained.

### Decision Values

Various embodiments of the presently described systems and methods include a computer algorithm stored in one or more computing devices that computes a decision value for a given feature. The algorithm, referred to as the decisionValueo function, evaluates how well a feature contributes to distinguishing between different outcomes. The algorithm takes as input all volume measurements for a given feature across all samples and the corresponding outcomes for those samples. For a given featureIdx, the system maintains an array of volumes (sampleVolumes) indexed by sample and an array of outcomes indexed by sample. The data structure used to store this information may vary as long as it allows for efficient retrieval of volume data and outcome data. In some implementations, the inputs may be represented as a single array of tuples, where each tuple contains a volume measurement and the corresponding outcome for a given sample (e.g., decisionValue(sampleVolumes, outcomes)=>number).

A decision value is high when a combination of volumes can produce a good outcome prediction. A decision value is low when the predicted outcome is uncertain. The system implements the decision value pruning based on features that have high decision values, indicating that they are highly associated with the target outcome(s).

In some embodiments, a confusion( ) or error( ) function may also be utilized. A high confusion value indicates that a feature does not strongly differentiate between outcomes, while a low confusion value suggests that the feature provides clear distinctions. The system may prioritize features with low confusion values to improve classification accuracy. The relationship between decision values and confusion values is inverse, meaning a decision value may be converted into a confusion value by negation or subtraction from 1 and vice versa.

There are various algorithms for computing decision values, each designed to evaluate how much predictive information a feature contains regarding specified outcomes. The objective of these algorithms is to assess the extent to which each feature contributes to the overall classification process, enabling the selection of the most biologically and statistically relevant features for outcome prediction.

### Pruning

The feature space can be reduced by pruning features that contribute little to predicting the desired outcomes. In various embodiments, the system retains only the more or most informative features, such as those with decision values above a predefined threshold and/or confusion values below a certain threshold.

In many embodiments, multiple features are retained rather than selecting only the single best-performing feature for three reasons. First, individual features may be subject to measurement noise, and combining multiple features helps mitigate this variability. Second, biological sample preparation and analysis often fragment molecules, meaning that individual parts rather than whole molecules are detected. Third, biological pathways typically involve multiple interacting molecules, so no single molecule can fully represent a given process or condition.

An analyst may enter custom pruning criteria via a human-computer interface. The interface may enable the user to specify the number of features to retain, set a minimum decision value threshold, or define a maximum allowable confusion value.

### Selecting Biological Tissue Analysis for Particular Biological Outcomes

In some biological problems there may be many possible indicators for a particular set of outcomes. The set outcomes might be whether a disease condition is present or not, whether a therapy succeeded or failed, or other possible outcomes of interest (binary or non-binary). There are many possible choices for where indicators for these outcomes might be found. It is also possible the combinations of features from more than one set of indicators might be appropriate. The presently described systems and methods include techniques for selecting which tissue analysis processes will form the best indicators for a particular set of outcomes.

### Candidate Analysis Processes

There are a variety of choices for finding relevant markers for a particular set of outcomes. These choices are grouped as follows:
(i) Tissue sample
(ii) Mass-spectrometry or RNA-seq
(iii) Tissue sample preparation
(iv) Chromatography column for mass-spectrometry
(v) Instrument settings for mass-spectrometry or RNA-seq

In the case of cancer, samples may include tissue samples from the tumor as well as from adjacent lymph nodes and perhaps the blood. One of these may be redundant, or one may be irrelevant. Ideally, a principled way to decide which samples should be included to provide the best information about the outcomes is utilized. There are multiple choices that can be used in each of these areas. If the blood can yield all the answers needed, that is useful to know because it is the least invasive and costly of the various sources.

For a given project, a target outcome set is selected that will focus on the questions of interest. The system can discriminate between large numbers of outcomes using correspondingly large numbers of samples. It may not always be feasible to try all possible combinations of choices among the possible preparation processes for the data. An informed choice can be utilized to narrow down on a few preparation choices.

### Selected Process

A selected process includes a tissue sample source, mass-spec or RNA-seq, sample preparation technique, instrument settings, etc. For an individually selected process, each of these must be the same or functionally equivalent across all samples. For a given individual organism, data can be collected using each selected process, and an outcome can be assigned to that individual. For each individual A and each selected process P, a feature vector F_{A,P} can be produced as described above. For each process P, there can be a feature space FS_{P}, which contains a description of each feature and the process that produced it.

### Composite Feature Vectors

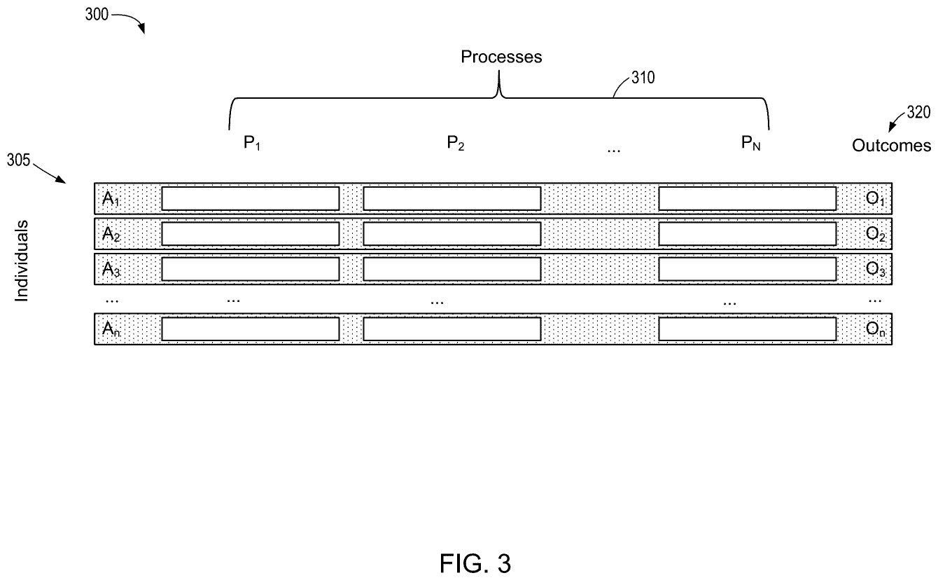

FIG. 3 illustrates a structured representation 300 of feature vector construction across multiple individuals, processes, and outcomes. Each row in the diagram corresponds to an individual (A₁, A₂, A₃, . . . , Aₙ) 305, and each column represents a specific process (P₁, P₂, . . . , Pₙ) 310 applied to the biological samples obtained from those individuals. The final column in each row corresponds to the observed outcome (O₁, O₂, . . . Oₙ) 320 associated with the respective individual.

A composite feature vector C_{Ai} can be generated for each individual be concatenating all the feature vectors Ai and processes Pj. This composite vector contains all the information from all the selected processes for that individual. A training set is generated by concatenating the composite vectors C_{Ai} with the outcomes O_{Ai} for each individual. A composite feature space can also be created by concatenating the feature spaces of the selected processes. Feature descriptors in the composite feature space include descriptions of the processes that they came from.

### Building Digital Assays Using Mass Spectrometry and DNA/RNA Sequencing

It is frequently useful to be able to separate biological conditions using simple tests. This might be separating patients who have a particular disease from those who do not or separating those who might respond well to a particular therapy from those who would not. Such problems can be characterized by a set of two or more outcomes. For example, separating occurrence from non-occurrence of a disease state and response from non-response to a therapeutic intervention. There may be more than two outcomes. For example, distinguishing four different stages of cancer plus the outcome of not having the cancer at all.

To create such assays, a training set created from a series of tissues samples drawn from subjects representing each of the outcomes is considered. Each tissue sample can be subjected to one or more preparation processes and/or analyses to produce a feature vector for each sample. These feature vectors can be combined with the tissue sample's outcome to form a training set. From such a training set, a machine learning algorithm can be applied to generate a classifier function. The classifier function can be applied to new feature vectors from new tissue samples to predict one of the outcomes. This classifier function forms the basis for a digital assay.

Once a classifier function has been trained, it is relatively cheap to apply it to any new feature vector. If many classifier functions have been trained for a variety of outcome sets, these classifier functions can form a diagnostic resource that can provide informative data about the conditions covered by the classifier functions.

### Assay Creation

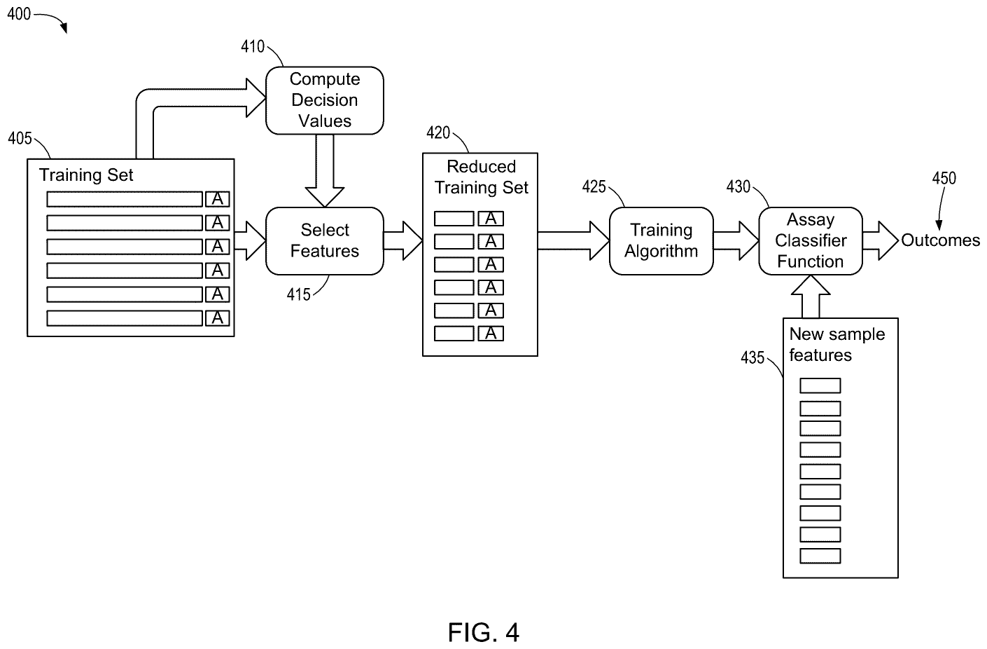

FIG. 4 illustrates a process 400 for creating an assay classifier function 430 by selecting the most relevant features from a training dataset 405 and using them to train a predictive model. The process begins with a training dataset 405, which includes multiple tissue samples collected from organisms representing each outcome in a given outcome set. Each sample undergoes one or more analytical processes to generate a composite feature vector based on, for example, molecular and biological characteristics. The feature vectors can be combined with the corresponding outcomes to form the initial training dataset 405 used for classification.

As previously described, one challenge in constructing a training set for biological assays is that the number of features often far exceeds the number of samples, sometimes by more than two orders of magnitude. To address this, the system computes decision values 410 for each feature. The decision value is based on the relevance of each feature to distinguishing between different outcomes. For example, each feature may be assigned a decision value between 0 and 1, or 1 and 100, or another range allowing for comparison. Examples of how to compute a decision value for each feature are described in greater detail below. The system then selects features, at 415, based on their decision values, retaining only the N most informative features (those with the highest decision values or the lowest confusion/error values), where N is an integer value or a small percentage of the original number of features. Alternatively, the system may select features, at 415, that have a decision value above a threshold decision value.

The resulting reduced training set 420 comprises only the N most relevant features identified via the feature selection, at 415. The reduced training set 420 is passed into a training algorithm 425, which develops an assay classifier function 430 capable of predicting outcomes based on feature vectors. Once trained, the classifier can be applied to new sample features 435, producing predicted outcomes 450.

### Feature Combination

In some implementations, only the best one or two features (highest decision value) are used from the feature selection and used as the classifier function. However, this may result in same shortcomings as current “biomarker” tracking, where a few indicators are used to understand a process. There are several problems with this minimal biomarker approach.

The first is that the features are generally not representative of full molecules and certainly not full biological pathways. Suppose there is a protein consisting of 300 amino acids. The mass spectrometry preparation and analysis process will break this down into peptides of less than 30 amino acids, with each peptide becoming a feature. This key molecule is now represented by more than 10 peptides, each potentially leading to a feature.

The RNA that produced the 300 amino acid molecule would consist of 900 nucleotide bases. A K=100 would generate 800 overlapping K-mers for this molecule. Thus, the RNA expression for this key molecule would exhibit many features. Frequently, multiple molecules impact different parts of a biological pathway. Each of these molecules could produce significant (e.g., relevant or salient) features to assist in creating an accurate assay.

In several studies, it has been found that the best features may have a decision value of only 0.9, meaning that they will be wrong 10% of the time. However, when many features (e.g., 10, 30, or 40+ features) are combined, assay classifier functions are created that approach 100% accuracy. Additionally, a single noisy feature can produce erroneous outcome predictions. Erroneous results from noise are mitigated by combining evidence from multiple features in making a decision (i.e., determining and outcome). In various embodiments, the machine learning training algorithm algorithmically combines and/or otherwise considers many features to produce more accurate outcome predictions than would be possible with only one or two biomarkers.

### Generalizable Classifier Functions

The presently described systems and methods include classifier functions that are accurate on samples that were not in the training set. Knowledge from the training set is generalized to the larger world. The presently described systems and methods avoid over fitting. Over fitting occurs when the classifier function does not capture the problem represented by the outcomes but rather memorizes the training set. An over-fitted classifier function does not behave well in the general case.

Over fitting is a particular problem in building digital assays because even after feature selection, there are frequently still more features than training examples. For many machine learning algorithms, a surplus of features and the limited number of samples leads to extreme over fitting. The presently described systems and methods address this problem by collecting information from all the selected features.

### Voting Classifiers

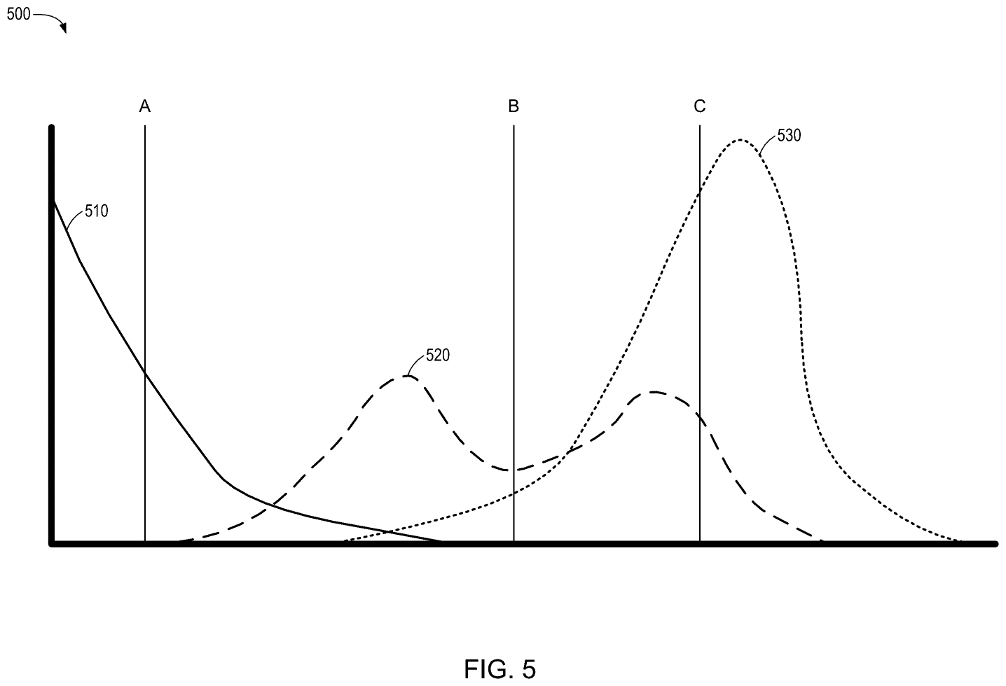

FIG. 5 illustrates a graph 500 that represents a voting classifier approach in which features contribute to outcome determination based on their decision values and confidence levels. The graph 500 shows distributions for three different outcomes, represented by the solid line 510, the dashed line 520, and the dotted line 530. The classifier evaluates sample points A, B, and C, which represent how the decision values impact classification confidence. Point A aligns strongly with the outcome of solid line 510, indicating high confidence in this classification. Point B is located in an overlapping region between distributions, which corresponds to a low confidence level because Point B does not distinctly align with any single outcome. Point C corresponds to the outcome of dotted line 530 but with moderate certainty based on the broader distribution at this value.

One approach to classification is to allow each feature to independently vote on the best outcome. In this method, every feature makes an outcome prediction, and the classifier aggregates these votes to determine the final result. While this approach can produce reliable classifications, its primary limitation is that all features contribute equally, regardless of their predictive strength. Features with low decision values have the same influence as those with high decision values, which may lead to misclassifications.

The presently described systems and methods enhance this approach by weighting each feature's vote based on its decision value. Features with higher decision values—which provide stronger predictive signals—are assigned greater influence in the voting process. This weighting mechanism ensures that features contributing clear and reliable outcome differentiation exert more control over the final classification.

The system incorporates confidence values into the voting mechanism. Instead of assigning equal influence to all features, the classifier considers the relative strength of each feature's association with a given outcome. By weighting votes based on both decision values and confidence scores, the system refines its predictions to provide a more accurate classification result.

### Therapy Prediction and Information Service

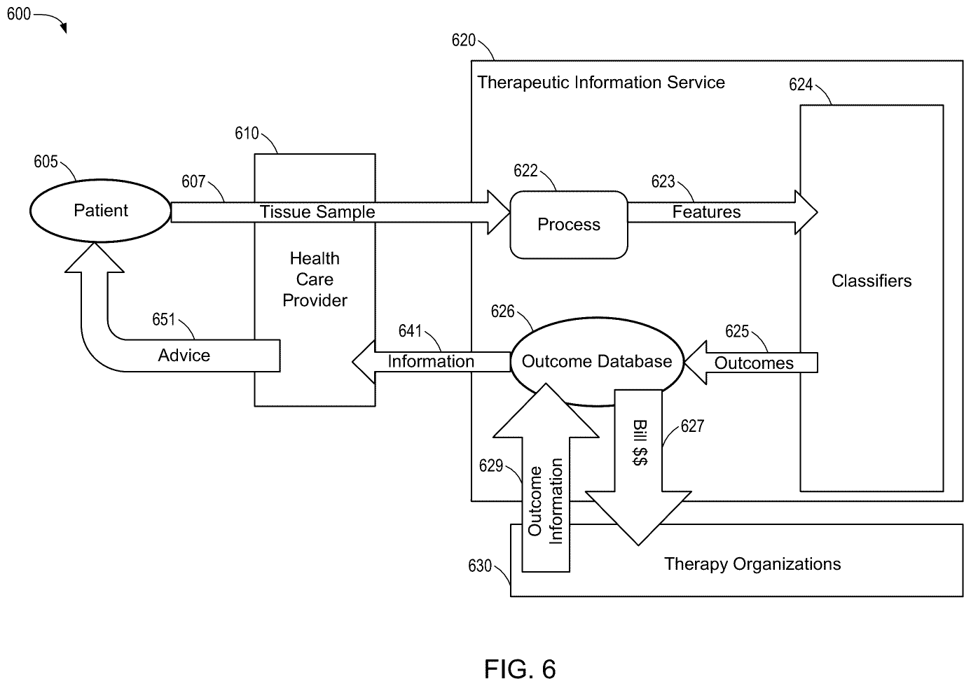

FIG. 6 illustrates a therapeutic information service system 600 to make therapy predictions and other decisions via the integration of patient data, outcome classifiers, and therapy organizations. As illustrated, a patient 605 consults a health care provider 610. The health care provider 610 collects a tissue sample 607 from the patient. This sample is processed, at 622, and analyzed through a therapeutic information service 620. The therapeutic information service 620 may utilize the systems and methods described herein, including the use of machine learning-based classifiers to predict potential therapy outcomes 625 based on, for example, molecular feature vectors extracted from the tissue sample 607. The processed sample generates features 623, which are passed to the classifiers 624 for evaluation.

A variation of the digital assay process described herein is therapy prediction, which aims to determine the effectiveness of a particular therapy T for a given condition C. Since therapy T may not always be effective, tissue samples 607 are collected from patients 605 before therapy is administered. The therapy results are then analyzed and assigned an outcome 625, such as success or failure. Machine learning techniques may be employed to create a classifier function, which uses pre-treatment tissue samples and therapy outcomes to predict whether a patient is likely to respond positively to the therapy.

If tissue samples are collected before and after therapy, the system can generate feature vectors representing each sample set, labeled as before and after therapy. By computing decision values and selecting relevant features from both pre- and post-therapy samples, the system identifies biomarkers associated with therapeutic response. These insights help refine therapy development and detect potential adverse effects and/or provide more personalized and/or more effective treatment strategies.

The therapeutic information service 620 may function as a centralized system that applies multiple classifier functions to patient samples. Over time, numerous classifier functions may be developed to predict various disease conditions and therapy outcomes. Since these classifiers are trained using consistent preparation and analysis processes, a single tissue sample 607 from a patient can be processed and evaluated across multiple classifiers with minimal cost beyond the initial sample preparation. The systems and methods described herein allow for rapid screening of thousands of potential disease conditions and/or therapy responses.

Therapy organizations or businesses with financial interests in specific therapy outcomes may integrate their classifiers (e.g., therapies, patient information, outcome information, etc.) into the system. For example, a company selling a therapy for a particular condition may be interested in identifying patients whose samples indicate a high likelihood of benefitting from that therapy.

As illustrated, the health care provider 610 submits a tissue sample 607. The Tissue sample undergoes a processing step 622 to extract features 623, which are then evaluated using multiple classifier functions 624. The resulting outcomes 625 are stored in an outcome database 626 and returned to the health care provider 610 as “information 641.” The healthcare provider 610 may use the information 641 to provide advice 651 to the patient 605. The therapeutic information service 620 may be configured to not store or process any personally identifiable patient information.

Therapy organizations 630 interested in specific outcomes can subscribe to receive notifications when relevant outcome information 629 is detected. For example, a business selling a therapy for a specific condition or disease may be interested in subjects yielding a positive outcome on that condition. Accordingly, when an outcome matches a therapy organization's criteria, the system attaches relevant information and forwards it to the health care provider 610. A billing process 627 allows the therapy organizations to pay for access to relevant patient outcome data. In various embodiments, the therapy organizations 630 do not receive direct access to patient identities. The health care provider 610 can assess the relevance of the therapy-related information and advises the patient on the most suitable course of action. In some embodiments, the economics may be reversed, such that the therapeutic information may service 620 may be interested in paying therapy organizations 630 to obtain desired outcome information.

### Machine Learning Features

In some embodiments, large molecules are fragmented into smaller components before mass-spectrometry analysis. Similarly, when creating K-mer features of DNA/RNA sequences, each K-mer represents only a portion of a longer nucleotide sequence. When a given large molecule affects a biological process, the detected features that correspond to the large molecule are based on the individual mass spectrometry features for the fragments of the molecule. Accordingly, the system may computationally or algorithmically reassemble fragment features into larger features that represent the whole or complete molecule more accurately.

### Feature Similarity

In some embodiments, the systems and methods implement a mass spectrometry analysis that detects molecules as fragmented components rather than in their original biological form. Large molecules, such as proteins, may be broken into smaller peptides (e.g., via trypsin digestion). During the mass spectrometry ionization process, molecules may undergo further fragmentation that results in charged molecular fragments. As described herein, the mass spectrometry data may represent or correspond to molecular fragments rather than intact biomolecules. The mass spectrometry separates molecules based on mass-to-charge ratio (m/z). Accordingly, chemically identical molecules that incorporate different atomic isotopes appear at distinct mass positions due to variations in atomic mass due to the different numbers of neutrons.

The separation of chemically identical molecules bearing different atomic isotopes depends upon the charge on the molecule because the system separates on mass/charge, not just mass. The system may utilize small quanta sizes for quantization to retain information. However, the system may cut measurements of the same molecule into different quanta based on differences in measurement. These factors distribute the signature of a given molecule across multiple features. Various embodiments of the systems and methods described herein reassemble the original molecule from the detected fragments, effectively putting this information back together.

Similar fragmentation issues arise in DNA/RNA sequencing. K-mer features represent short, overlapping segments of one or more longer nucleotide sequence. Because K values are typically much smaller than the full sequence length, high-decision-value K-mers may correspond to subsections of biologically significant genetic regions, and not to entire genes or transcripts. In addition, given that DNA and RNA encode for protein sequences, data connected to a given protein may appear in the mass-spectrometry data as well as the sequencing data. Various embodiments of the systems and methods described herein include combing these two evidence sources.

Features that are derived from the same source molecule type will tend to be highly correlated. If a source molecule is highly abundant then its various isotopic variants, fragments and possibly K-mer features will be similarly abundant. If a source molecule is scarce then its isotope, fragment and K-mer features may be similarly scarce. Sufficient information about the source molecule may not be available, but various embodiments of the systems and methods described herein are configured to find features that are correlated. By finding features that are correlated and combining them into super features, the system can tentatively reconstruct the information that was in the source molecule.

One complication is that two or more different source molecules may be highly correlated with respect to an outcome. This tight correlation indicates that these source molecules are tightly linked within the same biochemical process. Thus, the two molecules contain the same or similar information with respect to the outcome. Even if two features are correlated yet not actually from the same source molecule, they will both have similar contributions to the outcome decisions. Though both may have information of high value to the decision, the system may treat the information associated with each feature as being the same. The system may combine these correlated features to reduce the feature set without loss of decision ability.

Taking the feature set received from the decision value pruning step the system compares each feature against all other features and computes a correlation between them. The system may perform similarity checks after feature pruning because the pairwise comparison of features is order N² in the number of features N.

Given two features A and B, the system can extract the volume values for each sample to generate two arrays:

$$\begin{matrix}A [ sampleIndex ] = > volume \\ B [ sampleIndex ] = > volume\end{matrix}$$

According to various embodiments, the system may combine features A and B into a new combination feature.

### Pearson's Correlation

Using this data the system can compute a correlation value using Pearson's correlation:

$$Correlation ( A , B ) = \frac{N \sum {A}_{i} {B}_{i} - \sum {A}_{i} \sum {B}_{i}}{\sqrt{N \sum {A}_{i}^{2} - {( \sum {A}_{i} )}^{2}} \sqrt{N \sum {B}_{i}^{2} - {( \sum {B}_{i} )}^{2}}}$$

Pearson's correlation varies between 1.0 and −1.0. The system may ignore Anti-correlation (negative values) because they do not reflect the molecular and quantization effects being reassembled.

The system selects a feature F and creates a feature list of F plus all other features Cᵢ such that:

$$Correlation ( F , {C}_{i} ) >= threshold$$

The threshold is a minimum correlation value for combining features. The system may utilize threshold values between 0.9 and 0.98. The resulting list of features can be combined by summing their volumes to produce a single combined feature value.

The system may apply the combination step to all features, remembering which features have already been combined and not considering them again. This results in a set of “super features” where each super feature is a list of quantum and/or K-mer features. Some lists may contain only one quantum or K-mer feature that did not correlate well with any other feature.

### Feature—Example Vector Distance

Given two features A and B the system, according to some embodiments, computes an example vector for each that contains the feature volumes for each example. The vector represents how a given feature is expressed across the sample data. The system may identify salient features as those features that are similarly expressed across the same set of samples. Depending on the biology represented by two features A and B, the range of their volumes may be very different. The system may, for example, normalize these vectors before comparison. As an example, the system may implement a normalization process that includes dividing each vector by its Euclidean length. In such embodiments, each vector has the same length as the original vector but still varies in its expression level for each example. In other embodiments, the system may implement a normalization process that includes dividing all elements of the vector by the sum of the elements in that vector. In such embodiments, each vector has a total volume of 1.0 with each feature varying according to its original expression.

Once the vectors are normalized, the system may compute a distance between them using any of a wide variety of distance functions, as described herein. Two vectors with a very small distance are very similar. If the distance is below a threshold value, the system may combine the two vectors.

### Distance Functions

One distance function for comparing features is the cosine distance, which comprises a computation of the cosine between the two vectors. If two vectors are perpendicular (share no similar information) the cosine will be 1.0. If two vectors are identical the cosine will be 0.0. The system computes the cosine by doing a vector multiplication of two vectors, each divided by their length, expressible as:

$$Cosine ( A , B ) = {\sum }_{n = 1}^{N} ( \frac{{A}_{n}}{l e n ( A )} - \frac{{B}_{n}}{l e n ( B )} ) .$$

As described herein, the system may combine pairs of features with low cosine distances. Another distance function that the system may utilize is the Manhattan distance, which is expressible in terms of normalized vectors A and B as:

$$Manhattan ( A , B ) = {\sum }_{n = 1}^{N} \vert {A}_{n} - {B}_{n} \vert .$$

The Manhattan distance is the sum of the absolute value of the differences between corresponding elements of A and B. The system may combine features whose normalized vectors have a small Manhattan distance.

Another distance function that the system may utilize is the Euclidean distance, which is expressible in terms of normalized vectors A and B as:

$$Euclidean ( A , B ) = \sqrt{{\sum }_{n = 1}^{N} {( {A}_{n} - {B}_{n} )}^{2}} .$$

In some embodiments, the square root is omitted as it does not change the relative distances. Again, the system may combine features whose normalized vectors have a small Euclidean distance.

### Feature Combination

As previously described, the system may combine two features A and B into a super feature C. The element of C's example vector is the sum of the normalized vectors from A and B. The system then removes features A and B from the list of features and adds feature C to the list for further comparison. Adding feature C into the list for continued comparison can cause new composite super features to be created from other super features, combining even more feature information. A composite super feature may include a list of the feature descriptors as its component features. The value of the composite super feature is the sum of the normalized values of its component features.

### Selective Combination of Random Features

In some alternative embodiments, the system may combine features based on the decision values of their combinations. For example, the system may collect features in a heap sorted by decision values.

In this context, a heap may be, for example, a representation of a binary tree laid out in a one-dimensional array with indices starting at 1. For an item in the heap stored at location I, the two children of the item are at locations 2i and 2i+1, with the root of the tree at index 1. The parent of any item in the tree at location i is at floor(i/2). The system can use a heap to sort items such that Value(i)>=Value(2i) & Value(i)>=Value(2i+1). Accordingly, the value of an item is always greater than or equal to the value of both of its children. If these conditions are ever violated, the system may exchange a parent with its largest child, and the process repeats at the new location. The system may use this technique to correctly place a feature in a heap within log₂(N) steps, where N is the number of items.

The system may create each of the original M features as a composite super feature of length 1, which contains only that original feature. The system computes the decision value for each such feature and sorts them into a heap. For a target of N resulting composite features, the system may only consider the first N items in the heap, which are now the features with the best decision scores.

The system can randomly choose two features from the top N features in the heap and combine them into a new composite super feature. The system may compute a decision score for this new feature, insert the new feature at the bottom of the heap, and propagate the feature using a heapsort algorithm. If the feature is better than any of the top N features (e.g., it has a higher decision score), the feature will now be included in the top N, causing another of the features to drop out. The system may continue the random combination of pairs of composite features until the system stops finding any better features. The system may utilize this approach to include combination features because of their ability to contribute to better decisions, or where having equal decision values they combine more pieces of information (larger f).

The system may parallelize the processes described herein, including the combination and sorting processes described above. The same original features can be sent to P different processors and the selective combination algorithm can be run on each of them. Because the features selected at each step are random, each instance of the algorithm on each processor may produce different combination features. Each processor or thread can return its top N combination features which can be sorted to select an overall top N, and to remove duplicate combinations produced by different processors.

### Feature Reliability

As previously described, analysis via mass spectrometry and sequencing may generate tens of thousands to hundreds of millions of features. In various embodiments, the system selects those features that contain the most information about an outcome or outcomes of interest. The system may utilize statistical significance computations to confirm that the features selected are the best. For example, the system may compute the probability of the results being due to chance. A statistical significance with a P value of less than 0.0001 is normally considered outstanding. If the system computes the probability for 1 million features, then the probability is that the information from 100 of those features is just random chance.

Statistical significance also has a problem in that it relies on data that conforms to a known parametric distribution. The most common distribution is Gaussian. The feature data generated by mass-spectrometry and DNA/RNA sequencing is frequently non-Gaussian. Additionally, the different processes involved in the preparation and analysis of this data can impact the quality. With over 1 million features, doing a complex analysis to develop a statistical model may be prohibitive.

Various embodiments of the presently described systems and methods utilize the analysis algorithms themselves for evaluating the features relative to random chance. The system may confirm whether the decision value is due to randomness or an actual reflection of the biology being measured by that feature. Because there are so many variables in processes and in the computation of decision values, it is difficult to establish the role of randomness analytically. Given a set of tissue samples with outcomes, a preparation/analysis process and a method for computing decision values the system can establish a base for randomness in decision value determination. According to various embodiments, the system calculates a decision value for each feature as described above. The system then selects the N features that have the highest decision values.

The system may randomly select outcomes from the outcome set and apply them to the features. That is, the connections between an outcome and its corresponding feature are scrambled or shuffled. The system recalculates decision values using these random outputs. In some embodiments, the system repeats it R times and retains the highest decision value for each feature. The system selects N features from this second calculation that have the highest decision values. These may or may not be the same features previously selected. The system may increase the values of R to produce a more accurate representation of randomness in the outcomes. The system can set an arbitrary value for R or can repeat the second step for a feature until that feature's maximum decision value stops changing.

First, the system may identify N features and their decision values, which are referred to as the data decision value set. The system may then select N features and their decision values, which are referred to as the random decision value set. The system may use the random decision value set to evaluate the reliability of the data decision value set. Because the random decision value set was created using the same data and analysis techniques as the data decision value set, their decision values are comparable.

### Evaluating Reliability

In some embodiments, the system may evaluate the reliability of the data decision value set by taking the largest of all decision values in the random decision value set. This is referred to as the random decision value base. The system can improve the data decision value set by removing all features with decision values that are less than the random decision value base. This process may be referred to as maximum random base thresholding.

Maximum random base thresholding is a conservative approach. Maximum random base thresholding assumes that the random decision value base is uniformly probable across all features. The random decision value base is a worst-case analysis for decision values, but it is also highly improbable.

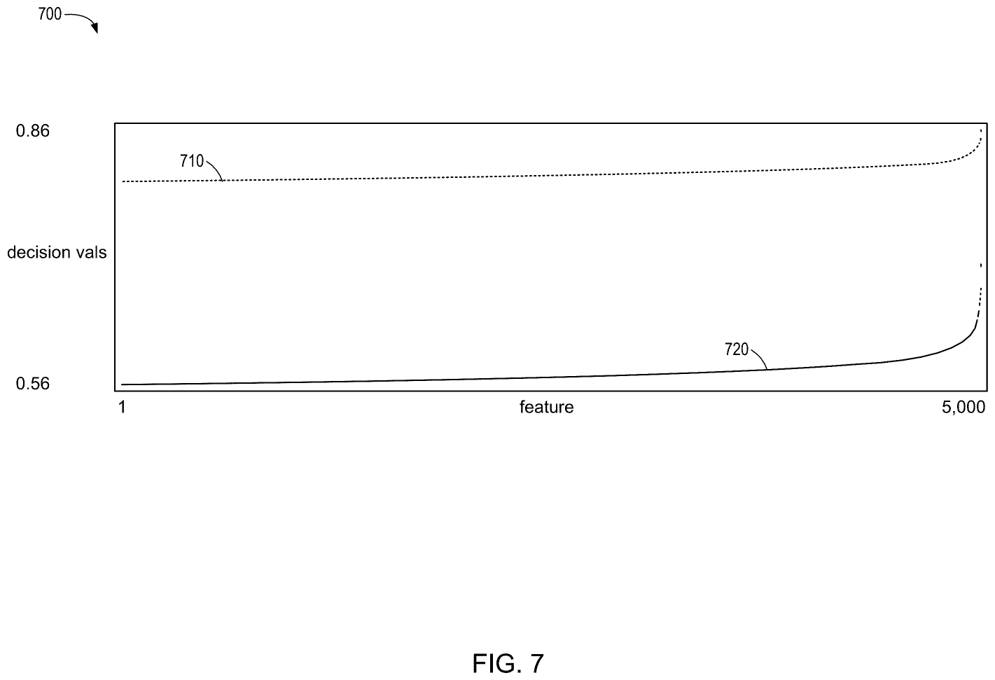

FIG. 7 shows a graph 700 of an example data decision value set 710 against the corresponding random decision value set 720. The horizontal axis corresponds to the features sorted in ascending order by decision value, and the vertical axis is the decision value. In the illustrated example, there are 5,000 features in each set. The random decision value base is 0.70. The data decision value set 710 (top line) varies between 0.80 and 0.86. Looking at the graphed line for the random decision value set 720 (bottom line), it is evident that the random decision value base is actually quite rare, even among the top N features. Many of the decisions from features use multiple features to mitigate the problem of an erroneous feature causing incorrect outcomes. In some embodiments, the system may accept a 1% probability of randomly measuring bad values for a feature. The system may randomly consider the top 50 features in the random decision value set 720. In the example, the top 50 features in the random decision value set 720 have decision values ranging from 0.62 to 0.70. This means that less than 99% of the features are expected to have a random base lower than 0.62.

If the system selects some random error probability of R, and there exists a random decision value set with F features, the system may determine the threshold by removing the top R*F features and taking the maximum of the remaining decision values. This threshold decision value is referred to as the probable random base. The system can use the probable random base to threshold the data decision value set 710 to confidently ensure that the remaining feature decision values are above random.

### Identifying Molecules of Interest

As described herein, the system may generate decision values for features using mass spectrometry and subsequent processing of feature vectors and training sets. Each feature represents a quantum range in mass charge (m/z) vs retention time (rt) space. The system may determine and use the mass and retention time of a molecule. Additionally, the system may determine and use the chemical identity of the molecule (e.g., the chemical composition and structure). The system may identify the molecule using MS-2, MS/MS (more likely LC-MS/MS) or mass-spectrometry stage 2. The system may use the MS-2 process on [m/z, rt] regions of interest. The system may utilize various algorithms and databases to select which part of the [m/z, rt] space, as first analyzed using MS-1 analysis (MS or LC-MS), should be processed further using MS-2.

While there are advantages to MS-2 analysis, conventional approaches present various challenges, including the following. First, conventional approaches devote significant instrument time to MS-2, meaning the MS-1 data is sparse when MS-2 is applied and when instrument run time is not extended. Second, conventional approaches tend to focus on molecules already known and ignore molecules that are not yet known. Third, conventional approaches tend to focus on high-abundance molecules and less on those of lower abundance. Fourth, conventional approaches spend a lot of MS-2 time on molecules that are not highly correlated with the outcomes under consideration.

The reliance on identifying only known molecules significantly limits the discovery of novel biomarkers that could impact outcome predictions. This approach is akin to searching only where light is already shining rather than where valuable insights may actually be found. Many disease-associated molecules are not necessarily highly abundant. For example, a tumor representing less than 1% of the body's mass is unlikely to produce highly abundant biomarkers in circulation. However, low-abundance indicators may still be present in detectable quantities. One of the most significant costs in mass spectrometry is instrument time, and MS-2 analysis greatly increases runtime and resource consumption if MS-1 data is not preserved for samples where MS-2 is performed. In various embodiments of the presently described systems and methods, the system prioritizes MS-2 analysis for molecules most strongly correlated with specific biological outcomes rather than applying it indiscriminately. This targeted approach enhances biomarker discovery, diagnostic precision, and therapeutic insights.

### Selecting Molecules for Further Study

Using techniques described above, the system can select those features that have high decision values. As described above these features are most indicative of the outcomes selected to consider. For each feature, there exists an [minMZ, maxMZ, minRT, maxRT] rectangular region in [m/z, rt] space. There are several ways to convert such regions into information about the molecules in that region.

Given a collection of reference data items were each item has at least a value for M/Z, a value for RT, and at least one more information item about molecules, the system may be configured to assume that this data was created in a manner consistent with the process used to create the features, as described herein. For a given feature F with region [minMZ, maxMZ, minRT, maxRT] the system can evaluate each item in the reference data collection to see if its M/Z and/or RT values fit within feature F's region. If it does, the system can add the information from that data item to the description of feature F and present it to the analyst through a human-computer interface. There may be multiple items in the data collection that match a specific feature, and one or more of them may be reported.

The data collection can come from any number of sources, including published databases, specialized algorithms, previous MS-2 analysis of similar samples and/or other analysis processes that yield information about molecules. For example, a given preparation/analysis of a given type of tissue sample might be used to test a wide variety of outcomes, with the analysis of these features included in the data collection. Previously identified features can then be used to inform the user about the identity of the features in their future experiments.

Without loss of generality, but with potential loss of accuracy the system may, in some embodiments, omit analysis with respect to RT and rely only on M/Z. The system may be configured to omit analysis of RT and rely on M/Z in instances when there is more variation in RT than in M/Z. Embodiments utilizing this approach will tend to have more data items that match a given feature. In such embodiments, data developed from one preparation/analysis process may inform a different preparation/analysis of the same tissues with some loss of accuracy in the identification. An amino acid sequence represents a protein or peptide of a given molecular mass. It is also possible to predict the probability of isotopes of such molecules and their mass. In some cases, the approach can yield salient data collection of proteins from a genome. The system may include information identifying those feature regions that cannot be found in the data collection in the report provided to the analyst, as they may represent molecules that are relevant to the outcome but warrant further study.

### Guided MS-2

According to various embodiments, given an MS-1 process, the system may generate feature vectors and training sets for a set of tissue samples and their corresponding outcomes. In such embodiments, the system may identify the top N features most relevant to these outcomes, along with their associated feature regions. Using these feature regions, the system may process the samples through an MS-2 analysis, targeting only the [m/z, rt] regions identified as biologically significant. This approach contrasts with general-purpose algorithms that select regions without considering their direct relevance to the problem at hand.

The system may integrate the data obtained from this outcome-focused MS-2 analysis into a data collection for future reference, as described in the Data Lookup section. To further optimize MS-2 processing time, the system may selectively perform MS-2 only on features from the N best list that do not already appear in the existing data collection. This targeted approach eliminates redundant analysis of known molecules, significantly reducing processing time while maintaining analytical accuracy.

When performing MS-2 focused on a set of feature regions, the system can apply MS-2 to each of the sample preparations used in the initial MS-1 analysis. This can be redundant. For MS-2 analysis, the system may only ensure that the feature regions identified for further analysis are found in reasonable abundance in at least one sample. Having identified the N features of interest, the system can identify which features are found in abundance in which samples. The system can make a list of the N features (FtoS). The system can select the sample that contains the most features from FtoS in sufficient abundance. The system can then remove those features from FtoS and repeat the process. The system remove those features from FtoS and repeats the selection process until all molecules of interest are accounted for within a smaller subset of samples. This refined sample set undergoes MS-2 analysis, focusing on the N best features.

In some embodiments, the system can further speed the MS-2 process by taking fluid from each of the original tissue samples and create a combined fluid sample that contains molecules from all sources. The system may process the combined fluid sample using MS-2, targeting only the [m/z, rt] regions corresponding to the N best features (e.g., those with the highest decision values).

### Improving Resolution of Quantum Features

In various embodiments, the system converts mass spectrometry data into features for analysis by identifying quantum features. Each quantum feature represents a rectangular region in m/z and RT and are defined by minimum and maximum values for both m/z and RT. The system may determine the width of each quantum feature along the m/z and RT dimensions. If the widths are too small, molecular information spread across m/z and RT may become fragmented into multiple features. This may result in an overwhelming number of data points and/or reduce analytical efficiency. Conversely, excessively large widths may group multiple molecular signals into a single feature, which can cause a loss of resolution and specificity.

In many cases, quantum widths are too broad to capture the fine detail typically observed in mass spectrometry data. To address this, various embodiments incorporate interactive computer graphic techniques that allow users to explore and refine the representation of quantum features for more accurate data interpretation.

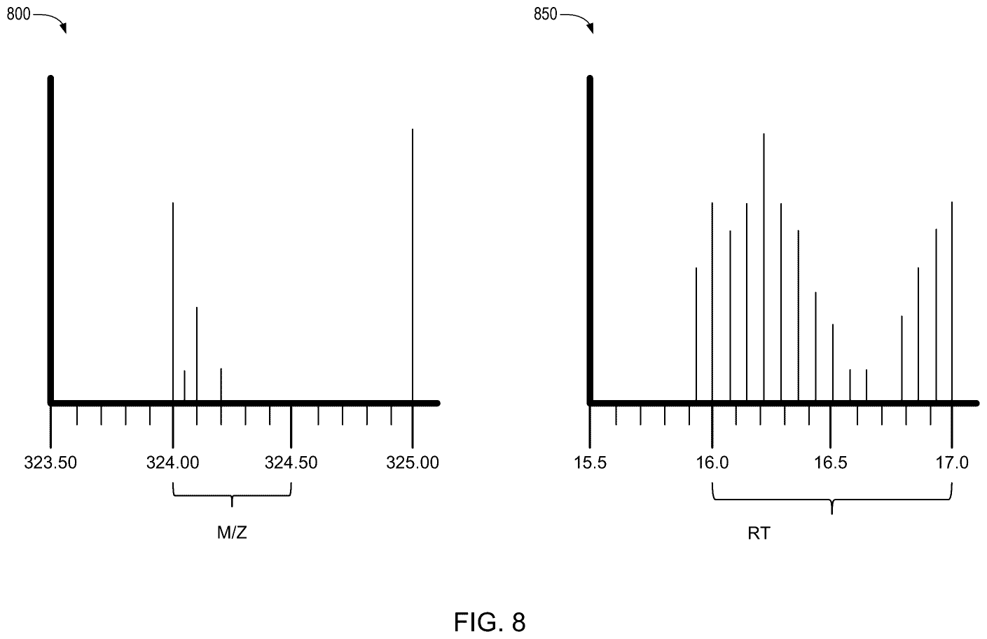

FIG. 8 shows a first spectrograph across the m/z dimension (m/z spectrograph 800) and a second spectrograph across the RT dimension (RT spectrograph 850), according to one embodiment. The system may use a quantum width of 0.5 daltons for m/z, which separates peaks at 324.00 and 325.00 but combines much of the information between 324.00 and 324.20. The system may use a quantum width of 1.0 minutes for RT, which captures most species peaking at 16.22 but misses data at 15.94. This setting also merges information from species that begin peaking at 17.0.

In the illustrated embodiments, all information within a quantum in each of m/z spectrograph 800 and RT spectrograph 850 is summed together. Accordingly, most of the information displayed in the spectrographs is lost. If the system reduces the m/z width to 0.02, all or at least more detail in the m/z spectrograph 800 would be preserved, but it would have 25 times the number of quanta, and the information about the peak at 324.00 would be spread among four different quantum features. Similarly, if the system reduced the RT width to 0.1, it would capture most or all the detail in the RT spectrograph 850 but would highly fragment the information about the region peaking at 16.22. The data between 15.9 and 16.7 likely originates from a single molecule. Additionally, using quantum widths of 0.02 for the m/z width and 0.1 for RT width, the total number of features is increased by 250 times, making interactive data analysis presentation more complex or unusable.

Using larger m/z and/or RT widths results in some loss of detailed information within a given quantum region; however, the system retains and captures key features. The challenge lies in accurately interpreting spectral data. In the RT graph, for example, a quantum width of 1.0 would obscure evidence of a new molecule emerging at 16.78.

As described herein, the system may compute a decision value for each quantum feature based on the data found in the examples. Using these decision values, the system can prune the feature set down to the N most decisive features, as described above. If N is set to 100, increasing resolution by 250 times would still result in a manageable 25,000 quanta. However, parsing the input data discards this high-resolution information.

### Requantization of Data

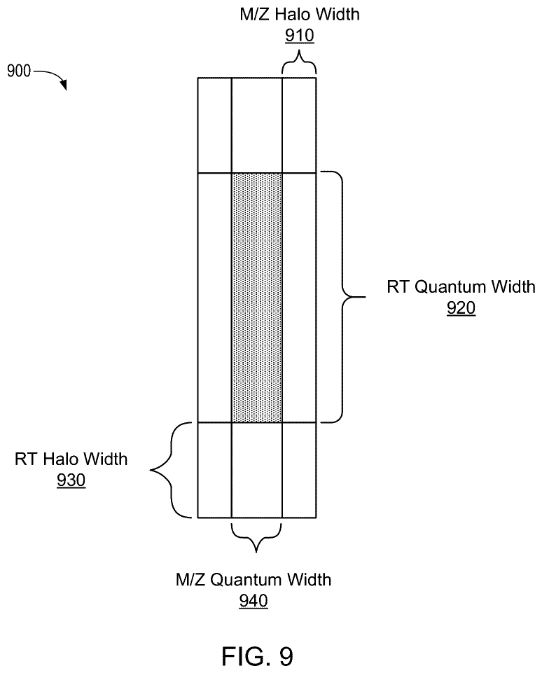

FIG. 9 illustrates a quantum rectangle 900 with an m/z quantum width 940 of 0.5 and an RT quantum width 920 of 1.0. A halo rectangle has been defined in terms of an M/Z halo width 910 and an RT halo width 930. The halo rectangle captures the region around the previous quantum region, which captures information that may have been cut off when the original quantum was defined, such as the data at RT=15.92 in FIG. 8.

For example, a halo rectangle may be specified with an M/Z quantum width of 0.5, an M/Z halo width of 0.25, an RT quantum width of 1.0, and an RT halo width of 0.5. The system may define new sub-quantum widths with an M/Z sub-quantum width of 0.02 and an RT sub-quantum width=0.1. The system may use the sub-quantum widths to subdivide the halo rectangle into sub-quanta. Given the values specified above, the halo rectangle is 1.0 in M/Z and 2.0 in RT. Given the sub-quantum widths, the halo rectangle is divided into 50 sub-quanta in M/Z and 20 sub-quanta in RT, resulting in a total of 1,000 sub-quanta for each original quantum feature.

In various embodiments, the system may compute a new sub-quanta for every quantum feature in the selected N-best features. In the case of the example above, the system may generate 1,000 sub-quanta for each original quantum feature. If the number of original quantum features, N, is 100 (i.e., N=100), the system defines 100,000 new quantum features as a high-resolution feature set.

The system (and/or analyst) may select E examples from the original data. The E examples may include the complete set of examples or a representative subset of the original examples. For each example E, the system may parse the original mass-spec data thereof. If an item of observed data falls within one of the sub-quanta in the high-resolution feature set, the system adds its abundance to the corresponding sub-quanta feature. If the observed data does not fall within one of these sub-quanta, the system may discard the data. In such embodiments, the system creates a new high-resolution feature vector for each example.

For each original quantum feature, the system generates a corresponding set of sub-quantum features that provide higher-resolution data. Given an example A from the selected set of E examples, the system may create a high-resolution display of the sub-quantum features for a specific original feature F. The system may generate the display by determining the maximum and minimum values across all sub-quanta derived from F. The system may assign a min-color and a max-color, interpolating colors based on each subquantum's value relative to this range.

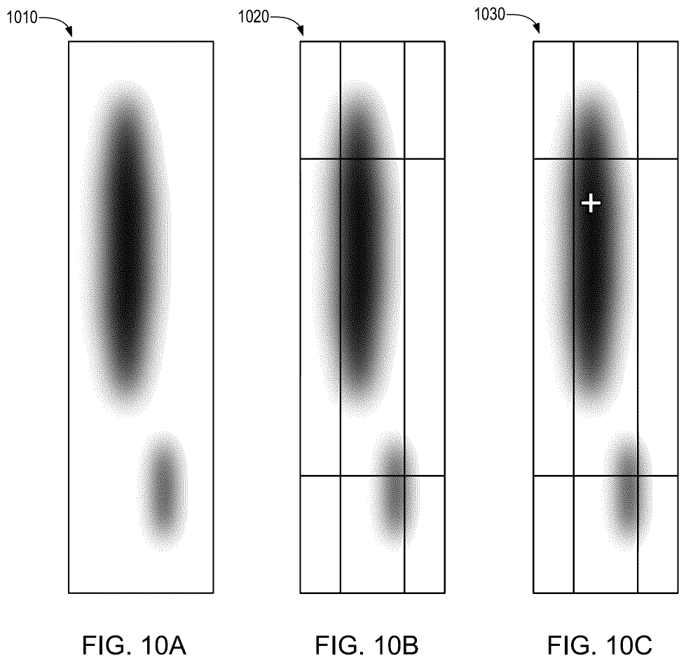

FIG. 10A illustrates an example color mapping 1010 of a sub-quantum region of example A and feature F, according to one embodiment. In the illustrated example, the system uses the color mapping to visually render or visualize sub-quantum regions using white for the minimum value and black for the maximum value in a given example A and feature F.

FIG. 10B illustrates an example color mapping 1020 with the boundaries of the original quantum region overlaid on the original color mapping 1010 of FIG. 10A.

FIG. 10C illustrates an example color mapping 1030 with the boundaries of the original quantum region and an additional highlighting or marker that identifies the sub-quantum with the maximum value, according to one embodiment.

The visualizations of FIGS. 10A-10C reveal that the original quantum feature of the single example A contains two distinct molecular signals. In some embodiments, the system may also sum sub-quantum values across all E samples and generate aggregate displays. In some embodiments, the system may separately aggregate values for each outcome and generate distinct visual representations, given that the data typically originates from a training set with predefined outcomes.

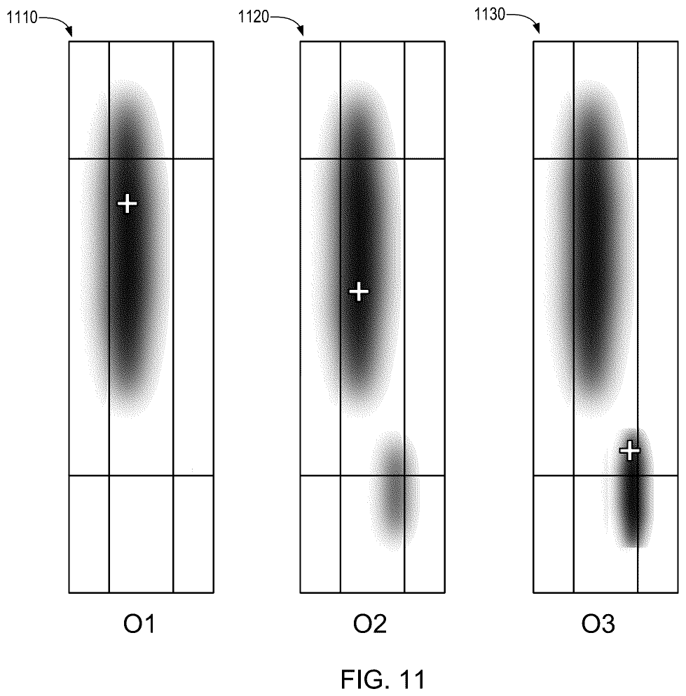

FIG. 11 illustrates a color mapping 1110 for outcome O1, a color mapping 1120 for outcome O2, and a color mapping 1130 for outcome O3, according to one embodiment. As illustrated, each illustrated color mapping 1110, 1120, and 1130 includes overlaid quantum region boundaries and a marker identifying the sub-quantum with the maximum value. Notably, the smaller molecule is absent in outcome O1 in the color mapping 1110, but reaches its highest abundance in outcome O3 in the color mapping 1130.

The sub-quanta displays in FIGS. 10A-C and FIG. 11 do not provide the kinds of views of the data shown in FIG. 8. Accordingly, in some embodiments, the system may project data for a slice of an original quantum feature.

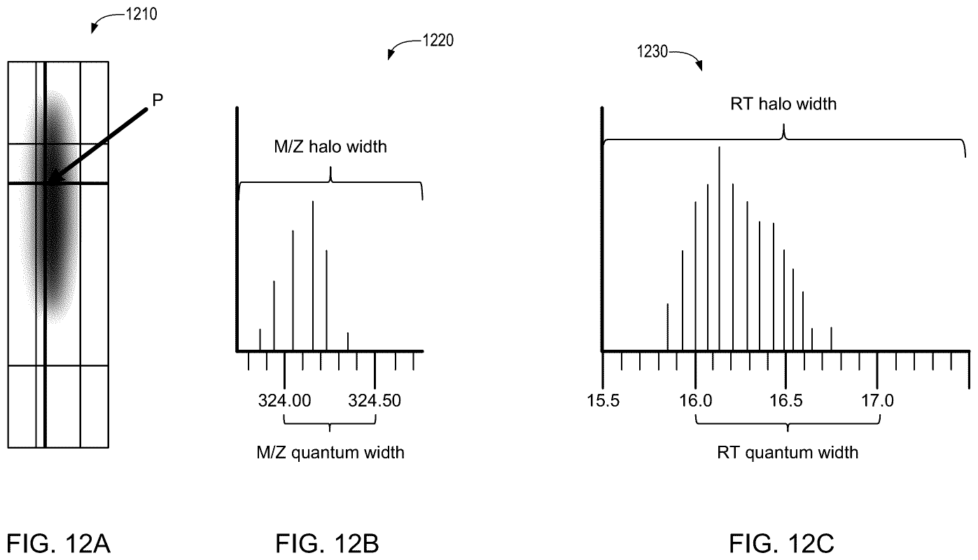

FIG. 12A illustrates a rendering 1210 of a particular feature for one example or the sums of a set of examples, according to one embodiment. The system may display the rendering 1210 for visualization by an analyst. The analyst may designate point P (e.g., using a mouse, pen, touch interface, or some other locator input device). The displayed horizontal line through point P represents a slice of all sub-quanta that have the same m/z as point P. The displayed vertical line through point P represents a slice of all sub-quanta that have the same RT as point P.

FIG. 12B illustrates an m/z graph 1220 of the values of the sub-quanta in the horizontal slice through the point P in the rendering 1210 of FIG. 12A, according to one embodiment.

FIG. 12C illustrates an RT graph 1230 of the values of the sub-quanta in the vertical slice through the point P in the rendering of FIG. 12A, according to one embodiment.

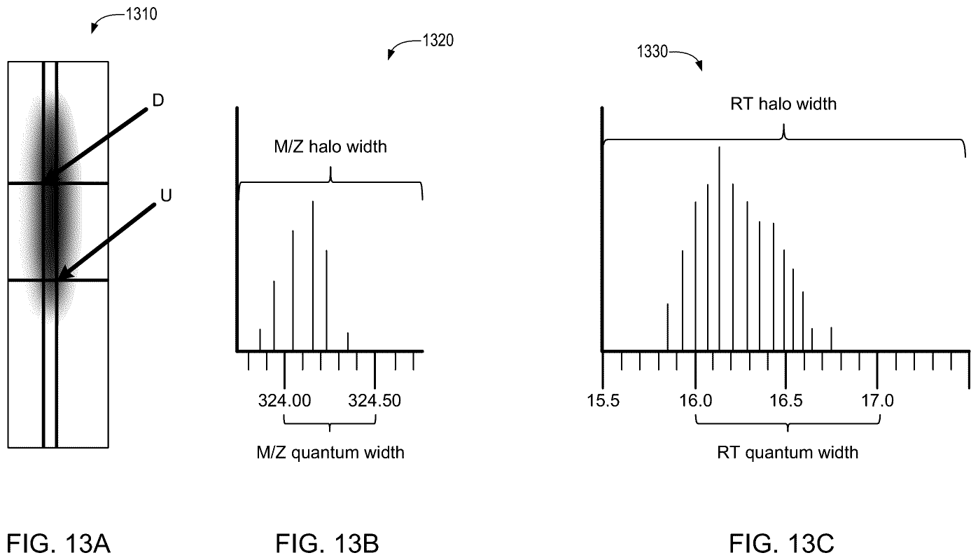

FIG. 13A illustrates a rendering 1310 of a particular feature or sum of set of examples, according to one embodiment. As illustrated, the analyst may specify two points D and U on the rendering. In some embodiments, the analyst or other user may make two selections, one for each of points D and U. In other embodiments, the analyst or other user may drag out a rectangle between points D and U. For the horizontal slice, the system may sum all the sub-quanta with RT values between D.RT and U.RT projected onto the M/Z axis by summing them across each M/Z value. Similarly, for the vertical slice, the system may sum all the sub-quanta with M/Z values between D.MZ and U.MZ.

FIG. 13B illustrates a graph 1320 for the horizontal slice associated with summed M/Z values based on the selected points D and U in FIG. 13A, according to one embodiment.

FIG. 13C illustrates a graph 1330 for the vertical slice associated with summed RT values based on the selected points D and U in FIG. 13A, according to one embodiment.

According to various embodiments, the system may generate renderings, graphs, charts, color mappings, and the like (such as those shown in FIGS. 10A-13C) for display via a graphical user interface for all N features or for a selected subset of E features.

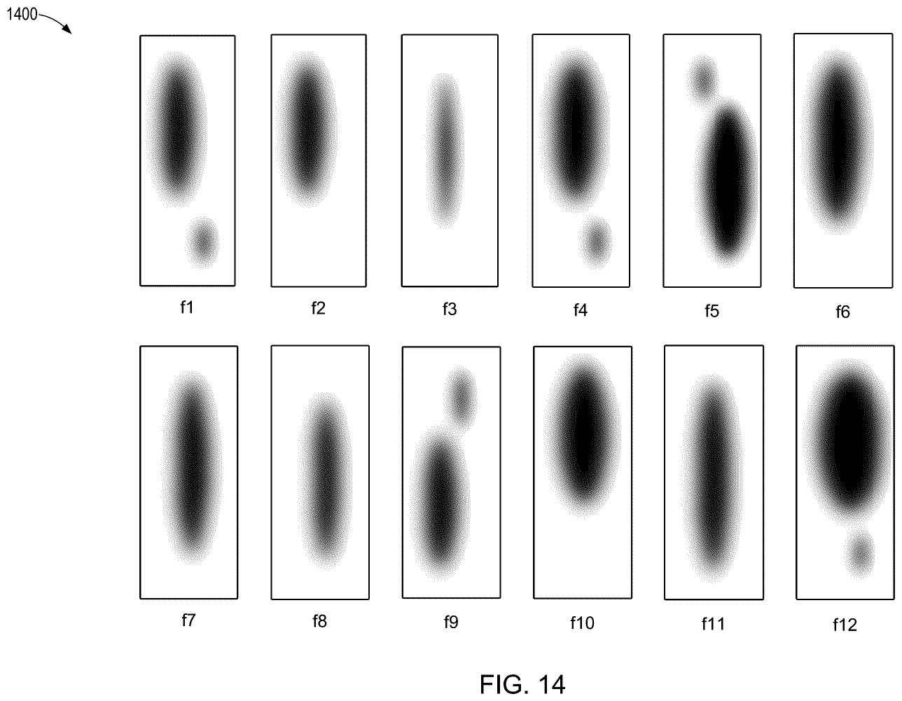

FIG. 14 illustrates color mappings 1400 for each of features f1-f12, according to one embodiment. Each of the color mappings 1400 may be generated as described above in conjunction with FIGS. 10A-13C and/or the other embodiments or combinations of the other embodiments described herein.

As previously described, an analyst or other user may select a point P within any one of the displayed color mappings 1400. The system may respond by generating new renderings for display via the graphical user interface for the selected feature with slice display graphs similar to those shown in FIGS. 12B and 12C. Similarly, the system may detect an analyst or other user input defining two points D and U (or a drawn box). In response, the system may render slice display graphs like those shown in FIGS. 13B and 13C for the selected feature (f1-f12).

According to any of the variously described systems and methods described herein, the system may identify the underlying molecules that influence or are influenced by a given condition or therapy. For example, if a patient has disease condition C for which the system is directed to develop a therapy, the system may identify what molecules and/or genetic information separates people who have the condition (D) from those who do not (D′). Given a therapy T for the condition C, the system may identify the biological consequences of the therapy T. For instance, the system may analyze the patient before the therapy T and after the therapy T to identify what has changed biologically.

In various embodiments, the system may derive feature vectors from mass-spectrometry and/or RNA-seq analysis, as described herein, and use the feature vectors to identify biological differences. For example, the system may select those features that have a high decision value or low confusion value, as described herein according to various embodiments.

As described herein, both mass-spectrometry and RNA-seq analysis produce hundreds of thousands to many millions of features. The amount of information is not processable by humans, regardless of the number of humans attempting to work in parallel (because humans cannot share memories) and regardless of the amount of time given (because the lifetime of a human is not long enough to analyze and compare the many millions of features). As previously described, the vast majority of identified features are irrelevant to a given outcome. These irrelevant features relate to, for example, biological processes that are not impacted by or causal to the condition or outcome being considered. As described herein, embodiments of the presently described systems and methods prune or filter the number of features from hundreds of thousands or millions to generate a set of salient features with only a few tens or hundreds of features that are identified as being the most salient to the problem being studied (e.g., the outcome or condition under consideration).

In some embodiments, the system constructs a training set using feature vectors derived from mass spectrometry and/or DNA/RNA reads, as described herein. Each feature vector is associated with an outcome from a predefined outcome set. The system evaluates each feature's decision value, identifying those that strongly differentiate among outcomes. The system may generate a salient feature set that only includes the features with high decision values that, for example, exceed a predefined threshold, rank among the top N, or represent a defined percentage of the highest-ranking features in terms of decision value. Extensive analysis and experimentation have shown that beyond the top 100 to 1,000 features, the other features (hundreds of thousands or millions of other features) have decision values that approximate random guessing,

The systems and methods described herein may calculate a decision value for each feature using a single decision value calculation approach and/or as a function (e.g., a weighted average) of the decision values calculated using more than one decision value calculation approach. Examples of decision value calculation approaches include but are not limited to, parametric statistical tests, feature value partitioning, and equiprobable distributions.

### Parametric Statistical Tests

In some embodiments, the system may use a T-test from the field of statistics to determine if a conclusion represents an underlying cause or can be explained by random sampling. The system may use the T-test to, for example, evaluate two Gaussian distributions and calculate the probability that their difference in means arises from random variation rather than a true underlying distinction.

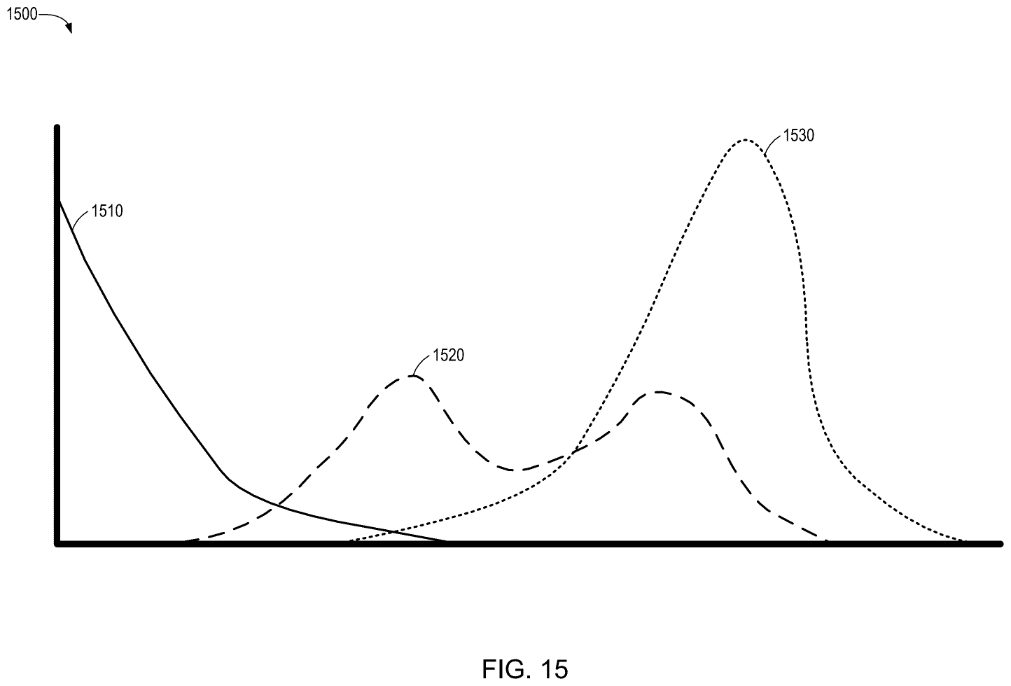

FIG. 15 illustrates a graph 1500 for three different outcomes, represented by the solid line 1510, the dashed line 1520, and the dotted line 1530. mass spectrometry and RNA sequencing data frequently produce non-Gaussian distributions. Because the T-test relies on the assumption that data follows a parametric model (most commonly Gaussian) applying it to these datasets can be challenging. While statistical models offer well-established techniques for handling parametric distributions, selecting and fitting the correct model for 100,000 to 100,000,000+ features can be impractical. Accordingly, the system may instead use non-parametric statistical tests, which provide a viable alternative for analyzing biological data.

### Feature Value Partitions

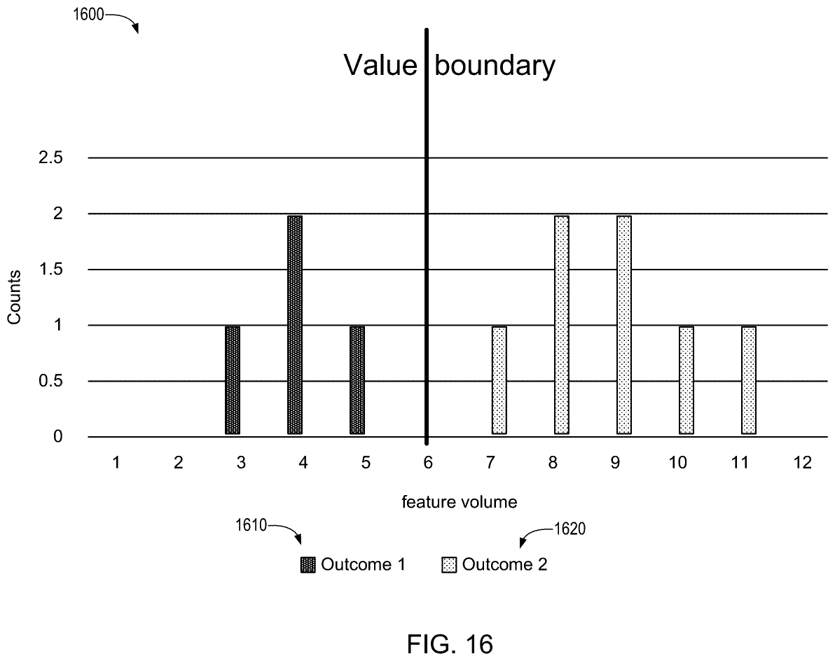

FIG. 16 illustrates a visualization 1600 of an approach to computing decision values using feature value partitions. A feature value partition consists of one or more value boundaries, which act as numerical thresholds to separate data into distinct partitions. In this example, the system applies a value boundary at six (6) on the horizontal axis to separate a first outcome 1610 and a second outcome 1620. Because this boundary perfectly divides the outcomes, it achieves a decision value of one (1), meaning it provides complete differentiation between the two groups.

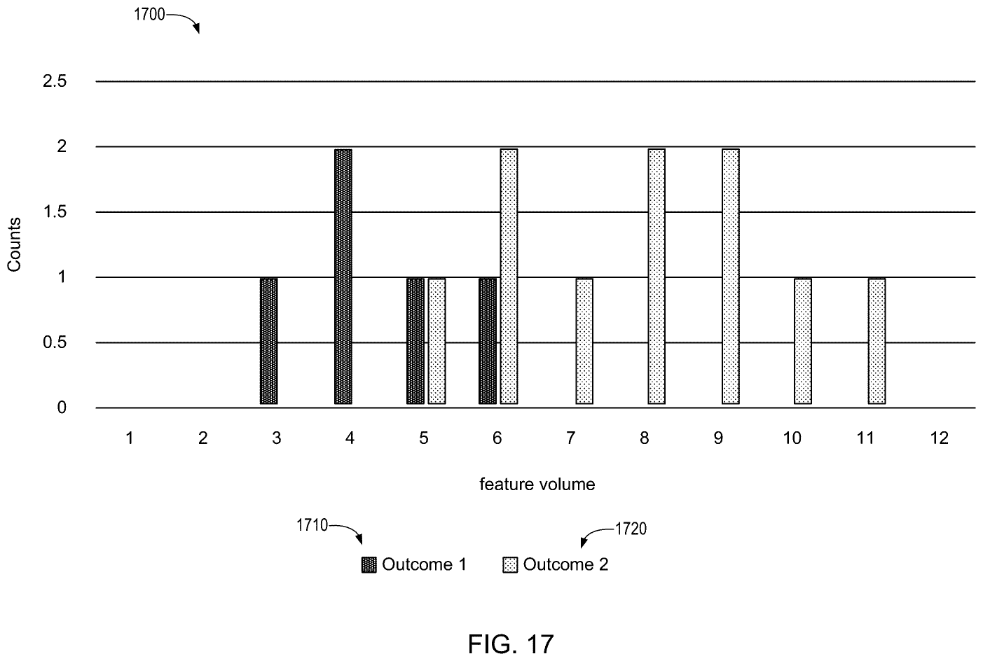

FIG. 17 illustrates visualization 1700 of situation where no single value boundary can perfectly separate the first outcome 1710 and the second outcome 1720. Regardless of where the boundary is placed, some data points from each outcome will be misclassified, making it impossible to achieve a decision value of one (1). Although the feature in FIG. 17 does not produce a perfect classification, it still provides useful information. A boundary placed anywhere between four (4) and seven (7) effectively separates the majority of the outcomes, though not all. When combined with other features in subsequent processing steps, this feature may still contribute to an accurate classification.

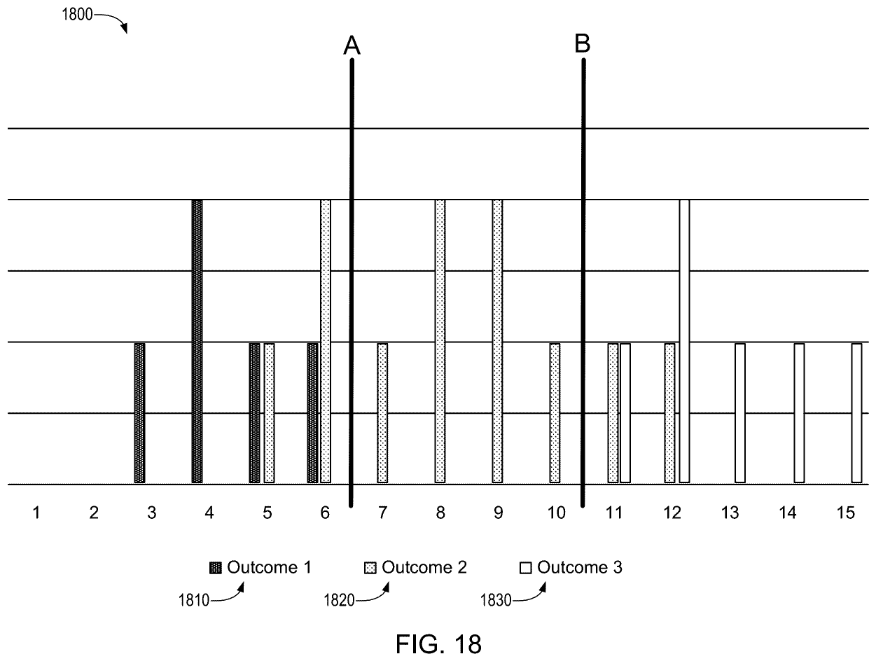

FIG. 18 illustrates a visualization 1800 of a scenario with a first outcome 1810, a second outcome 1820, and a third outcome 1830. The system may use value boundaries A and B to divide the feature into three partitions. Although each partition contains a dominant outcome, some overlap remains, meaning no single boundary can fully separate all three groups.

In various embodiments, the system generates feature value partitions with high decision values (or low confusion values) to maximize classification accuracy. As previously described, the system selects features based on the decision values to generate a salient feature set that includes the most informative features contributing to the final outcome classification.

The partition between boundaries A and B is “pure” because it contains only samples of the second outcome 1820. The partition is a perfect decision for the second outcome 1820. The partition to the right of boundary B has 5 samples of the third outcome 1830 and two samples of the second outcome 1820. Thus, this partition is less pure than the middle partition but still has strong evidence for the third outcome 1830. The partition to the left of boundary A has four examples of the first outcome 1810 and two examples of the second outcome 1820. This partition is the least pure but still has some evidence for the first outcome 1810.

A purity measure yields high values for sets that contain mostly one outcome and lower values for more mixed sets. In some formulations, an impurity measure is used. Impurity measures have low values for sets that have mostly one outcome and high measures of more mixed outcomes. Purity and impurity are converses of each other, such that one can be converted into the other by negation. Purity measures often, but not necessarily, have a maximum value of 1 and impurity measures often have a minimum value of 0. The system may operate to optimize for maxima (purity) or minima (impurity).

The system may apply purity and/or impurity measures to a set of outcomes. For example, the samples that appear to the left of boundary A correspond to a specific set of outcomes. The system computes the probability (Pₒ) of each outcome in the set. In the set to the left of boundary A, P₁=4/6=0.666 for the first outcome 1810. Similarly, P₂=2/6=0.333 for the second outcome 1820, and P₃=0/6=0 for the third outcome 1830. Similar probabilities can be computed for the other partitions. The system may determine one or more purity/impurity measures using the calculated probabilities. Examples of purity/impurity measure include, but are not limited to, information entropy, GINI purity, GINI impurity, and partition purity.

Information entropy measures the amount of randomness in a set. It can be used to form an impurity measure and is expressible as: Entropy=−Σ_{0=outcome} Pₒ*log₂(Pₒ). In some embodiments, the system uses the calculated entropy value as a confusion value or 1-Entropy as a decision value. GINI purity is a purity measure that returns 1.0 when a set is pure and 1/N when a set is mixed, where N is the number of outcomes. GINI purity does not require a computationally expensive log function, which is expressible as: GINIPurity=Σ_{o=outcome} Pₒ². In various embodiments, the system may use GINI impurity as a substitute for information entropy, where GINllmpurity=1−GINIPurity. In some embodiments, the system utilizes a measure in the form of GINIPurity, where any of a wide variety of impurity or purity measures are used, according to the conditions described above.

The system may determine decision values the measure of a partition instead of the measure of a set. Thus, the system may use the partitions to make decisions. That is, the system may use a measure of how good a partition is at deciding the desired outcome all by itself without considering other features. To compute this, the system may compute the purity (impurity) of each partition and then the weighted average of those purities based on the number of samples in each partition.

For example, the system may compute the GINIPurity of each partition. In the illustrated example, the purity of the left partition is 0.555, the purity of the middle partition is 1.0, and the purity of the right partition is 0.592. The purity of whole partition is (0.555*6+1*4+0.592*7)/22=0.675.

The system may not be provide with the partition for a feature. Instead, the system may select a partition for each feature using one or more partitioning techniques. For example, the system may take the maximum and minimum values for the feature and divide it into N equal partitions. This approach provides some indication of how well the feature divides the outcomes. For example, if the system takes the feature values in FIG. 18 and divides them into two partitions at 7.5, the resulting partitions provide good separation of the first outcome 1810 from the third outcome 1830, but a poor separation with respect to the second outcome 1820. Dividing the features into three partitions at 5 and 10 is better. The system may utilize this partitioning technique (partitions in halves, thirds, or other number of equally spaced partitions) because it is fast. If there are lots of examples relative to the number of partitions, this approach also provides a reasonable estimate of the decision power of the feature. A larger N or number of partitions will give better resolution to the decision but some partitions may end up with too few examples for any statistical significance.

In some embodiments, the system may use a sorting partitioning technique in which the system sorts the feature values and then divides them into N partitions with equal numbers of samples in each partition. This approach adapts the partitions to where the samples are actually distributed but pays little attention to where good boundaries are between outcomes. This approach may be slower because of the sorting process.

In some embodiments, the system may use a sort and test partition technique in which the system sorts the values and then applies successive value boundaries between values while testing the decision value of each boundary. This approach works especially well when there are only two outcomes.

In some embodiments, the system may use a mean-value partitioning technique in which the system computes the mean value for each outcome and sort those means. The system assumes that there is a good value boundary between each pair of means as they appear in sorted order. The system tests all possible boundaries between a pair of means to find the boundary with the highest purity (lowest impurity). The boundary selection with the highest purity is selected as the value boundary to separate the two means. This technique handles any number of outcomes by finding the best separation between means.

If the system receives a value for a given feature, the system can identify the partition that includes the value using an inequality test. The system may assign the outcomes to each partition that has the highest probability. The system may also assign a confidence value to each outcome/partition as the purity or 1-impurity of partition. The system may utilize the assigned information to evaluate the features relative to target outcomes, as described herein.

In various embodiments, as described herein, the system may compute a decision value for each respective feature by partitioning measured feature values into two or more intervals, and then quantifying how distinctly each interval separates the outcome categories based on the computed purity or impurity measure.

### Equiprobable Distributions

The feature partition techniques described above work on any distribution of outcomes in the data. However, the results produced are limited by the number of partitions and the subtleties of how two distributions might overlap may not be considered. In some embodiments, the system can regain some of the power of probability distribution models without the drawbacks of parametric distributions by approximating a probability distribution directly from the data.

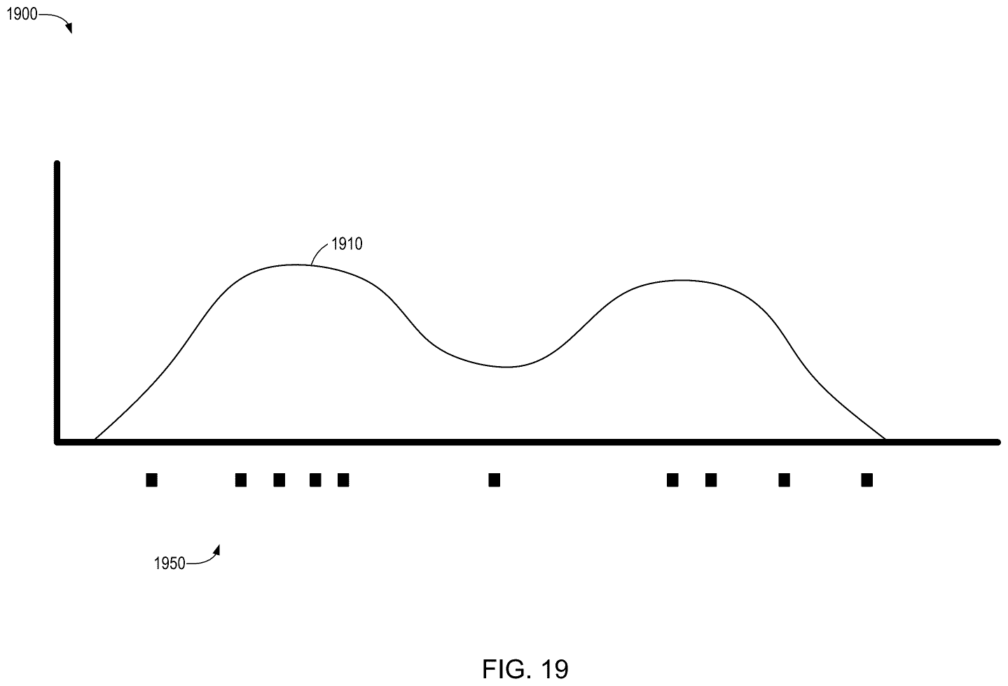

FIG. 19 illustrates a graph 1900 of a probability distribution 1910 and a set of data points 1950 drawn from the probability distribution 1910. The data points 1950 cluster more closely in regions of higher probability. As an example, N sample points may be selected in an unbiased manner and sorted. The probability of a sample point falling anywhere between two successive sample points is 1/N, which represents the probability of the entire region between point Pᵢ and point Pᵢ₊₁. The system may calculate the probability that a given point P belongs to the distribution using the formula: Probability(P)=1/N/(Pᵢ₊₁−Pᵢ). That is, the probability of a point in the distribution is determined by dividing the probability of the region by its width. probability is the probability of the region divided by the region's width.

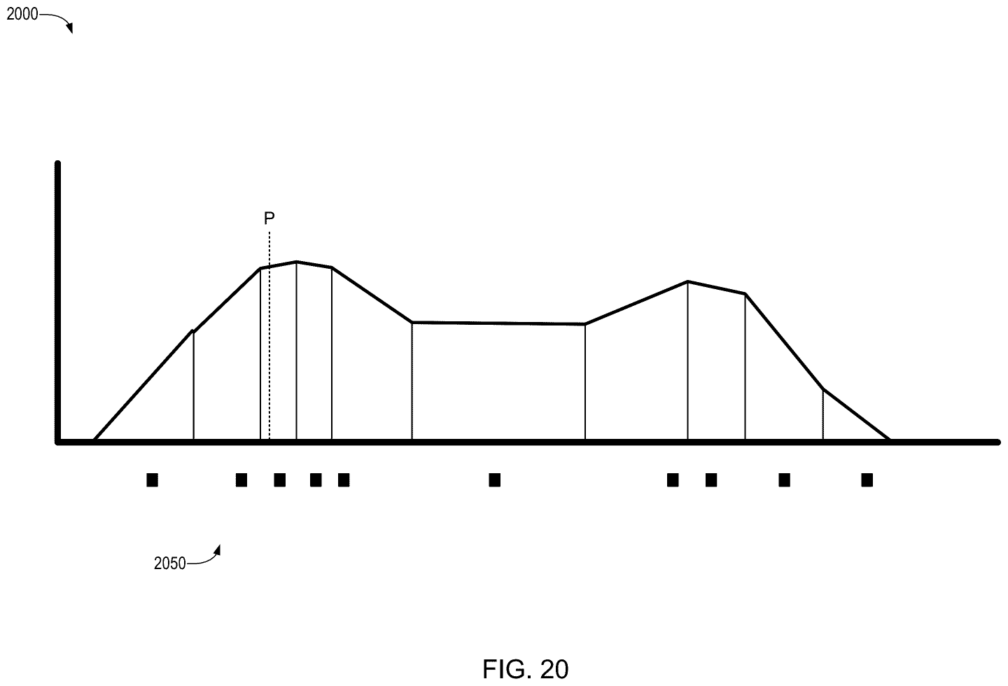

FIG. 20 illustrates an improved model 2000 for estimating probability distributions by refining the handling of the sample points 2050. Instead of treating all points between two sample points equally, the system replaces the sample points with midpoints between adjacent sample pairs. The system assigns each midpoint a probability using the same method as described above. For a new point P, the system interpolates between these midpoints to estimate its probability more accurately. Increasing the number of sample points enhances the approximation of the probability distribution. As more points are collected, the system refines the estimated distribution, reducing uncertainty and improving accuracy.

### Computing Decision Values

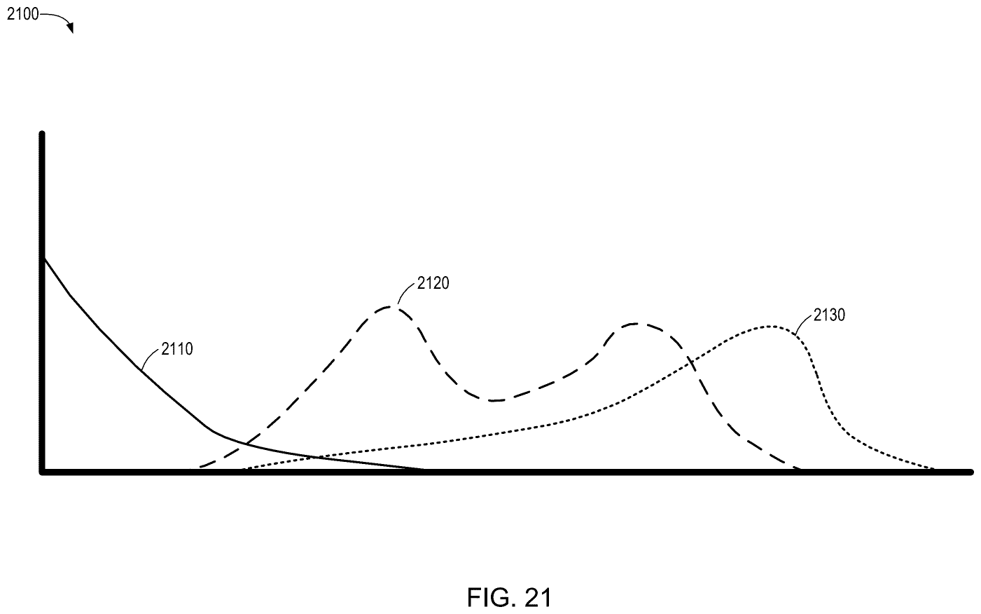

FIG. 21 illustrates a graph 2100 of probability distributions for a feature, F, with three distinct outcomes. The solid line 2110 represents the probability distribution for a first outcome, dashed line 2120 represents the probability distribution for a second outcome, and dotted line 2130 represents the probability distribution for a third outcome. The feature, F, is not pure due to significant overlap between the distributions. However, despite this overlap, the feature still provides useful information for distinguishing between the outcomes.

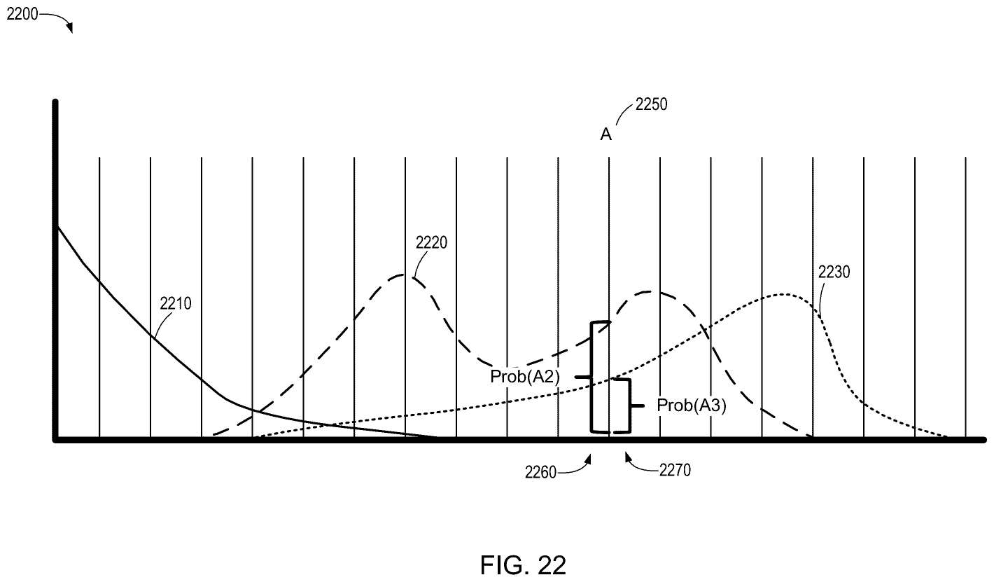

FIG. 22 illustrates the process 2200 of computing a decision value for a given feature, according to one embodiment. Again, the solid line 2210 represents the probability distribution for a first outcome, dashed line 2220 represents the probability distribution for a second outcome, and dotted line 2230 represents the probability distribution for a third outcome. As illustrated, the system may sample the probability distributions uniformly across the range of sample data to evaluate the feature's effectiveness in distinguishing outcomes. For a given point A 2250, the system determines the decision value using the highest probability among the outcomes. For instance, the system may associate the highest probability, Prob(A2) 2260, with the second outcome (represented by dashed line 2220) and the second-highest probability, Prob(A3) 2270, with the third outcome (represented by dotted line 2230).

According to various embodiments, the system calculates the value at point A:

$$Value ( A ) = ( Prob ( A 2 ) ‐ Prob ( A 3 ) ) / Prob ( A 2 )$$

If Prob(A3) 2270 were zero, then Value(A) would be 1.0. If Prob(A3) 2270 were half of Prob(A2) 2260, then Value(A) would be 0.5. If Prob(A3) 2270 were nearly as large as Prob(A2) 2260, then Value(A) would approach zero. According to such embodiments, the system measures how different the “best” outcome at a given point is from the “second-best” outcome. In some embodiments, the system may use the calculated Value(A) as a confidence value in the outcome.

The system may use the Value function to compute a decision value by averaging the values of all the sample points as follows:

$$DecisionValue ( F ) = \frac{{\sum }_{i = 0}^{N} Value ( {P}_{i} )}{N}$$

The system can process any number of sample points, as the computational cost is incurred only during training. Increasing N improves the accuracy of DecisionValue(F). However, setting N significantly higher than the number of original data points may create a false impression of greater accuracy.

### Separation of Outcomes

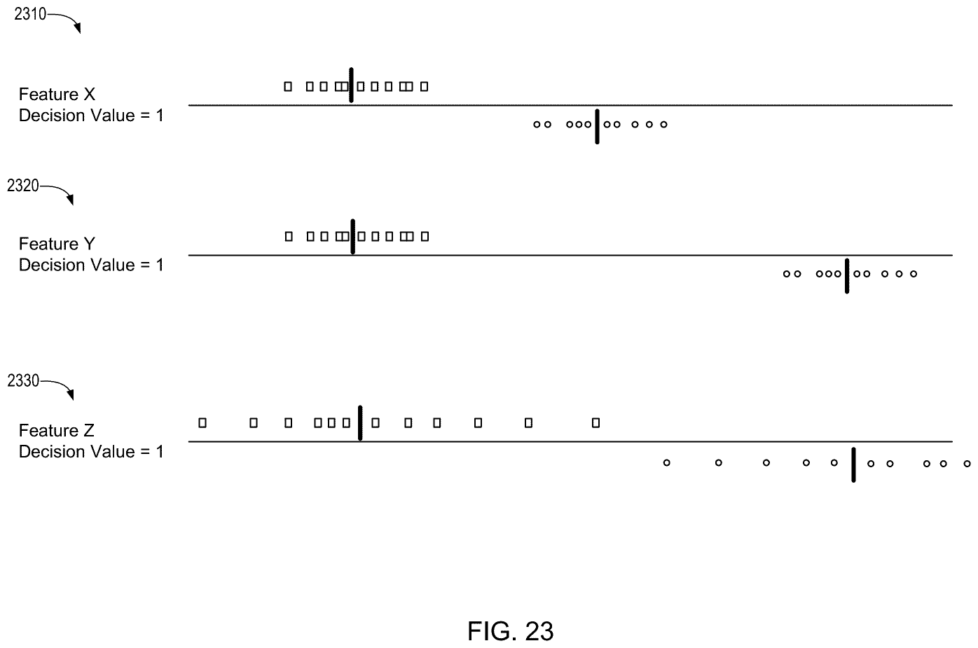

FIG. 23 illustrates Feature X 2310, Feature Y 2320, and Feature Z 2330 with sample data for two outcomes. Both the feature partition technique and the equiprobable distributions yield a decision value of 1.0 for all three features.

Feature Y 2320 provides better separation than Feature X 2310 because the separation is wider. The greater the separation, the more likely the feature is to contribute to an accurate decision. Although Features Y 2320 and Feature Z 2330 share the same mean values, Feature Y 2320 is a better choice because it exhibits a larger distinction between the distributions.

The gaps between outcomes of all the probabilities may be zero, and so the equiprobable Value function may be meaningless. This issue arises when the partition and equiprobable decision functions are 1.0.

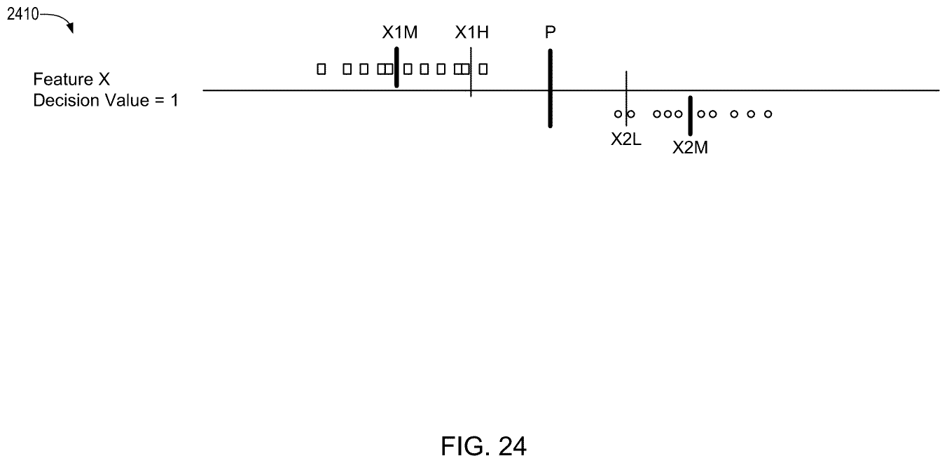

FIG. 24 illustrates additional data used to resolve this issue for Feature X 2410, according to one embodiment. In the illustrated embodiment, the data points are defined as follows: X1M is the mean value for the data points for Feature X, first outcome; X1H is the high value for the data points for Feature X, first outcome; P is a partition point to separate the first outcome from the second outcome; X2L is the low value for the data points for Feature X, second outcome; and X2M is the mean value for the data points for Feature X, second outcome.

The system may use X1H and X2L to determine the spread or width of the two outcome distributions. The system may set X1H as the maximum value of all points in X1 and set X1 L as the minimum value of all points in X2. However, using the max and min values can introduce errors if there are severe outliers. Alternatively, the system may set X1H at the 90th percentile and set X2L at the 10^{th} percentile. The percentile chosen may vary as long as they are uniform.

The system may compute point P as (X1H+X2L)/2, which is the midpoint between the extremes. The placement of P is not critical because there is no information in the gap to inform the placement. The system can compute the decision value of Feature X 2410 by first computing the spread of each outcome as Spread1=X1H−X1M, Spread2=X2M−X2L, and SpreadAve=(Spread1+Spread2)/2. The system may then calculate the decision value as DecisionValue(F)=1.0+(X2L−X1 H)/SpreadAve.

The system may add 1.0 so that the decision value will be greater than the decision values derived when the distributions overlap. The difference between the extremes indicates how far apart they are, and the division by SpreadAve normalizes this distance by the width of the distributions.

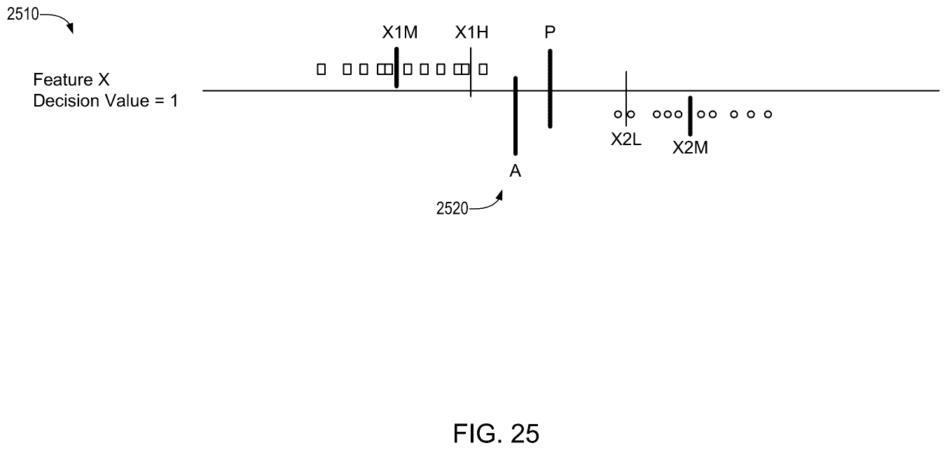

FIG. 25 illustrates an approach used by the system to compute an outcome and a confidence score for a given point A 2520 for Feature X 2510, according to various embodiments. In the illustrated example, if A<X1H, then point A 2520 is associated with the fist outcome with a confidence of 1.0. If A>X2L, then point A 2520 is associated with the second outcome with a confidence of 1.0. If A>=X1H and A<P then point A 2520 is associated with the first outcome with confidence (P−A)/(P−X1H). If A<=X2L and A>=P, then point A 2520 is associated with the second outcome with confidence (A−P)/(X2L−P).

Confidence values in the regions less than X1H and greater than X2L are dictated by the data, which has no confusion. Within the gap, the confidence value is based on the position of point A 2520 relative to the position of partition point P. Confidence values decrease linearly from 1.0 at the extremes of the distributions down to 0.0 at P where the decision becomes ambiguous.

### Selecting Tissue Analysis for Outcomes

In some biological problems there may be many possible indicators for a particular set of outcomes. For example, the set of outcomes might include whether or not a disease condition is present, whether a therapy succeeded or failed, or other possible outcomes of interest. There are many possible choices for where indicators for these outcomes might be found. It is also possible the combinations of features from more than one set of indicators might be appropriate. The system may utilize various techniques, as described herein, to select which tissue analysis processes will form the best indicators for a particular set of outcomes.

Tissue analysis processes used to identify relevant biological markers can be categorized into several groups, including tissue sample selection, mass spectrometry or DNA/RNA sequencing, tissue sample preparation, chromatography column selection for mass spectrometry, and instrument settings for mass spectrometry or RNA sequencing. Additional process variations and parameter selections may also influence the system's ability to detect meaningful biological markers for specific outcomes.

In the case of cancer, the system may utilize tissue samples from the tumor as well as from adjacent lymph nodes and perhaps the blood. One of these tissue sample sources may be redundant and/or one may be irrelevant. The system may utilize a principled and systematic approach to decide which tissue sample sources is/are salient to provide the best information about the outcomes. For example, if the blood can yield all the answers, that is useful to know because it is the least invasive and costly of the various sources.

For a given project, the system may choose an outcome set that will focus on the questions of interest. The system may utilize a large number of samples to discriminate among a relatively large number of outcomes. In many instances, it is not feasible to try all possible combinations of choices among the possible preparation processes for the data. Informed choice may be utilized to narrow down on a few preparation choices.

The system may define or select a process in terms of a tissue sample source, mass-spec and/or DNA/RNA sequencing analysis, sample preparation technique, instrument settings, etc. For a given individual organism, data can be collected using each selected process and an outcome can be assigned to that individual. For each individual A and each selected process P, the system can produce a feature vector F_{A,P}, according to any combination of the various embodiments described herein. For each process P there may be a feature space FS_{P} that contains a description of each feature and the process that produced it.

As described herein, the system may generate feature vectors for all individuals Ai and processes Pj. The system may generate a composite feature vector, C_{Ai}, for each individual by concatenating together all feature vectors for Ai. The composite feature vector, C_{Ai}, contains all the information from all the selected process, P, for that individual.

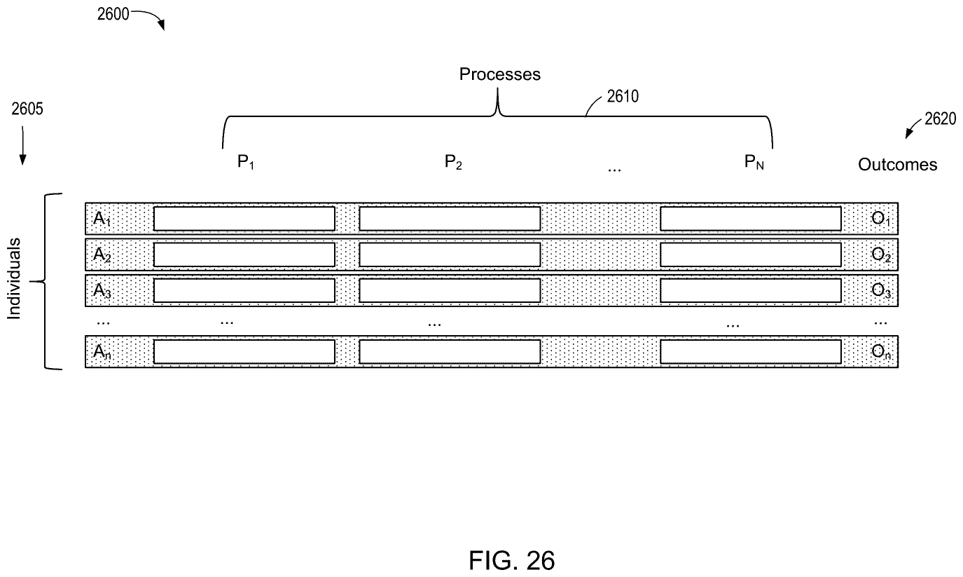

FIG. 26 illustrates a training set 2600 of composite feature vector, C_{Ai}, for all individuals 2605 and processes 2610 with outcomes, O_{Ai}, 2620. In various embodiments, the system may generate a composite feature space by concatenating the feature spaces of the selected processes 2610. Feature descriptors in the composite feature space include descriptions of the processes that they came from.

The system may use the training set 2600 to compute decision values for each feature in the composite feature space. The system may sort features by their decision value and identify the most important features relative to the outcomes 2620. The top N decision values correspond to those that are the most highly indicative of the outcomes, as previously described herein. In various embodiments, the system may use the top N features to identify the processes 2610 that are most associated with highly relevant features.

For example, the system may be used to analyze tissue samples prepared using four different preparation processes. Hundreds of thousands or millions of features may be originally considered. As described herein, the system may select 1,000 features with the highest decision values for inclusion in a salient feature set. The system may evaluate the relevance of each decision process based on the number of salient features associated therewith and/or the decision values of the salient features associated therewith.

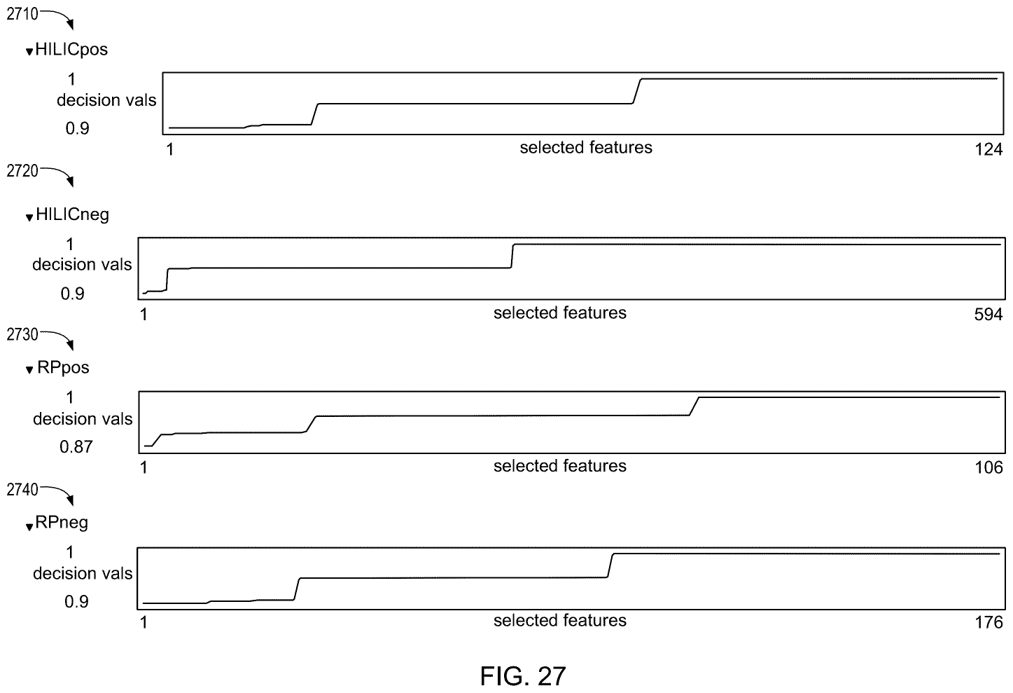

FIG. 27 illustrates the decision values of the features associated with each of four different decision processes, according to one embodiment. As illustrated, the decision values of the features are plotted with the decision values ascending to the right. The HILICpos process 2710 includes 124 features with decision values ranging from 0.9 to 1.0. The HILICneg process 2720 includes 594 features with decision values ranging from 0.9 to 1.0. The RPpos process 2730 includes 106 features with decision values ranging from 0.87 to 1.0. The RPneg process 2740 includes 176 features with decision values ranging from 0.9 to 1.0.

Notably, the HILICneg process 2720 accounts for 594 of the 1,000 best features. Over half of these have a decision value of 1.0 (perfect separation of the outcomes). This indicates that the outcomes could be completely decided using only HILICneg process. The other processes may be considered redundant and/or less valuable since they have fewer features and/or lower average decision values.

If the differences among preparations were not so pronounced, the system may focus on the best process(es) using machine learning and cross-validation. In some embodiments, the system may use each of the processes to train a machine-learning algorithm using only the feature vectors from each respective process. The resulting machine learning algorithm results can be measured using cross-validation or some other learning validation process. If none of the processes score 100% in cross-validation, training sets may be utilized that contain feature vectors from multiple processes. The approach described above may be used to determine the process or combination of processes that best decide the outcomes. In subsequent situations, redundant or less valuable preparation/analysis processes may be omitted. As described herein, model parameters of a classification system may be optimized or adjusted to reduce misclassification rates. For example, cross-validation techniques may be used to optimize the model parameters to reduce the misclassification rates.

### Tools for Understanding

Mass spectrometry and nucleotide sequencing data may be collected across a series of outcomes within a single parameter. For example, in a therapy study, blood samples may be drawn from patients before treatment, then at various intervals such as 2 days, 7 days, and 30 days post-treatment, or the like. This approach generates data representing four distinct outcomes within the parameter of time relative to therapy administration. Because these outcomes form an ordered series, understanding the biological effects of the therapy over time becomes critical. The methods described herein apply not only to therapy-related studies but also to any scenario involving a sequential progression of outcomes.

Many biological studies involve collecting samples in an ordered sequence, whether over time or through another structured pattern. Such scenarios can be represented as a training set with an ordered series of N outcomes, O1, O2 . . . ON, where N is three or greater. To draw out the progress of the biology across these outcomes, the system may identify N binary decisions (or N−1 binary decisions) to compare the outcomes. The system may construct binary decisions using any of a wide variety of approaches, including the two approaches described below.

In some embodiments, the system may use a compare-to-base approach. In such embodiments, the system considers Outcome O1 as the base against which all other outcomes are compared. The system constructs N−1 decisions O1-O2, O1-O3, . . . O1-On. Given these N−1 decisions, the system computes decision values for each feature/decision combination.

In some embodiments, the system uses a compare-to-previous approach. In such embodiments, for each outcome O1 through On−1, the system generates the decisions O1-O2, O2-O3 . . . On−1−On. The system compares each outcome to the previous outcome in the series. Given these N−1 decisions, the system computes decision values for each feature/decision combination.

Given the organization of binary decisions, the system calculates N−1 decision values for each feature. To select the best B features, the system may select a single decision value. In some embodiments, the system may use the maximum decision value across all decisions for a given feature. If one of the decisions turns out to be particularly easy, then that decision's decision values for each feature will generally be higher. However, choosing the top B features using this technique can drop out features that would be useful for more difficult decisions. However, taking the average decision value across all decisions for a given feature will water down features that are important for one particular decision.

To resolve these issues, for each decision D and each feature F, the system may create a list of tuples {D,F,decisionValue} and sort that list of tuples in ascending order by decisionValue. For each tuple, the system may assign an ascending index representing feature F's importance to decision D. Higher indices correspond to higher decision values.

The system can combine the indexed list of tuples for each feature and binary decision into a single list and sort by index. For each tuple T for feature F for which there is a tuple T′ for feature F such that T.index<T′.index, the tuple T is removed from the list. The system may identify one tuple for each feature F, specifically the one with the highest index. If T.index==T′.index, the system retains the one with the highest decisionValue. Because the system sorted on index rather than decision value directly, each binary decision's features have an equal chance of being at the top of the list. The system can now select the B features that are highest in the index-sorted tuple list. The system prunes the feature set to those features with all binary decisions fairly represented.

In working with trends across an ordered outcome series, the system uses a technique called parallel axes. The parallel axes technique uses a two-dimensional chart with a category axis and a value axis. The category axis can be the X axis of the chart with the value axis along Y, or X and Y can be reversed. The category axis is ordered but need not be numeric.

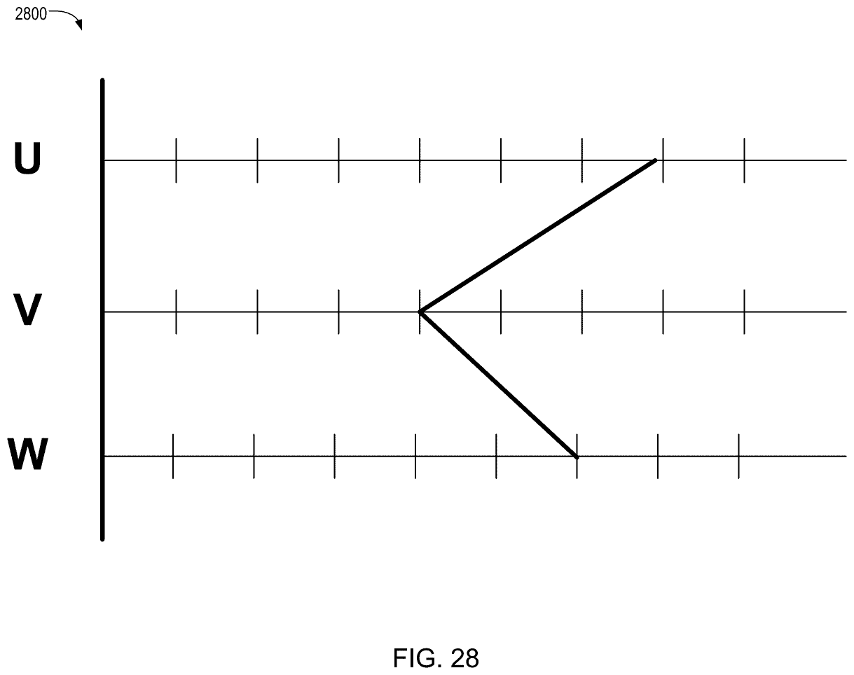

FIG. 28 illustrates a plot 2800 representing a feature F across an ordered outcome series, according to one embodiment. The feature has a value for each category, and its behavior is visualized as a line connecting its values in sequential category order. The plot 2800 is based on feature F having values F.U=7, F.V=4, F.W=6 for three categories U, V, and W. The system can plot many features in this way to compare how features display similar trends across the categories.

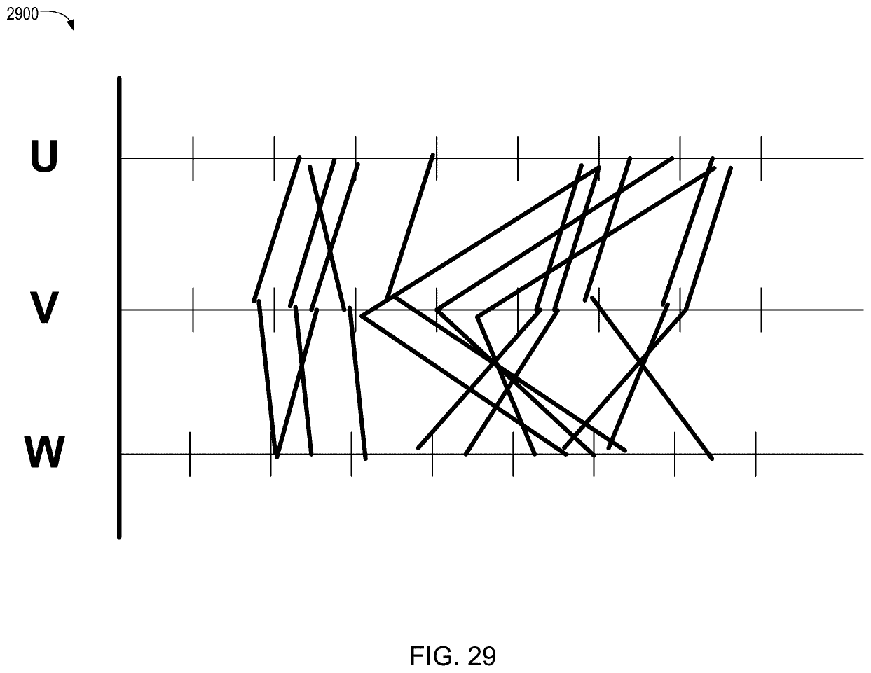

FIG. 29 illustrates a multi-feature graph 2900, where multiple features are plotted in parallel to identify trends across categories. By overlaying the trajectories of several features, the system can reveal patterns in how different features behave across an ordered series of outcomes. When analyzing datasets with hundreds of features, identifying meaningful trends can become complex, even when features exhibit similar behaviors.

The system's visualization techniques help mitigate this challenge by structuring the data in a way that facilitates pattern recognition and comparative analysis. To assist in understanding how features trend across the outcomes in an ordered series, the system adds interactivity to the display. The system may present a graphical user interface to facilitate analyst or other user interaction.

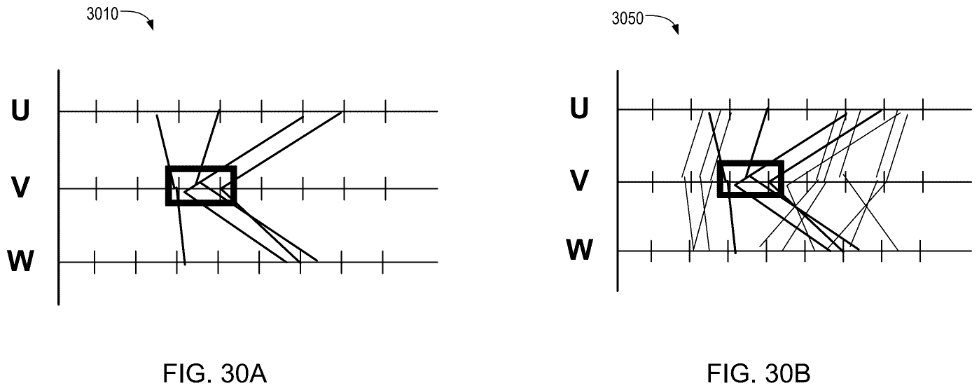

FIG. 30A illustrates a displayed graph 3010 in which an analyst has drawn a rectangle on the parallel axes display to remove unselected features. According to various embodiments, the system selects all features having a point within the drawn rectangle and removes any unselected features.

FIG. 30B illustrates a displayed graph 3050 in which the system has de-emphasized the lines associated with selected features, according to one embodiment. For example, the unselected features (or the selected features) may be presented in a different color, as thicker lines, with transparency effects applied, etc. In various embodiments, more complex selections can be created by adding additional selection rectangles, other selection shapes, or selection of specific lines.

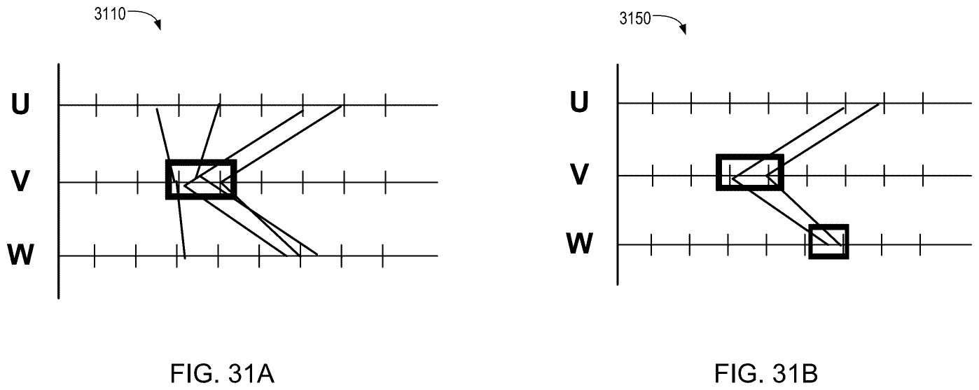

FIG. 31A illustrates a display 3110 with a first selection of features, according to one embodiment. As illustrated, a single rectangle is used to remove many of the lines associated with unselected features, as compared to FIG. 29.

FIG. 31B illustrates a display 3150 of two selections of features using two different rectangles. As compared to FIG. 31A, the new rectangular selection results in an additional three lines being removed. In various embodiments, the user interface allows for all features to be selected in response to a user adding a selection rectangle that does not select any features. Thus, if a selection rectangle is added that does not select any features, then all selection rectangles are removed, which makes all features selected. This technique allows an analyst or other user to select any empty space to reset the selection. According to various embodiments, the system uses parallel axes to create interactive charts for exploring trends in outcomes.

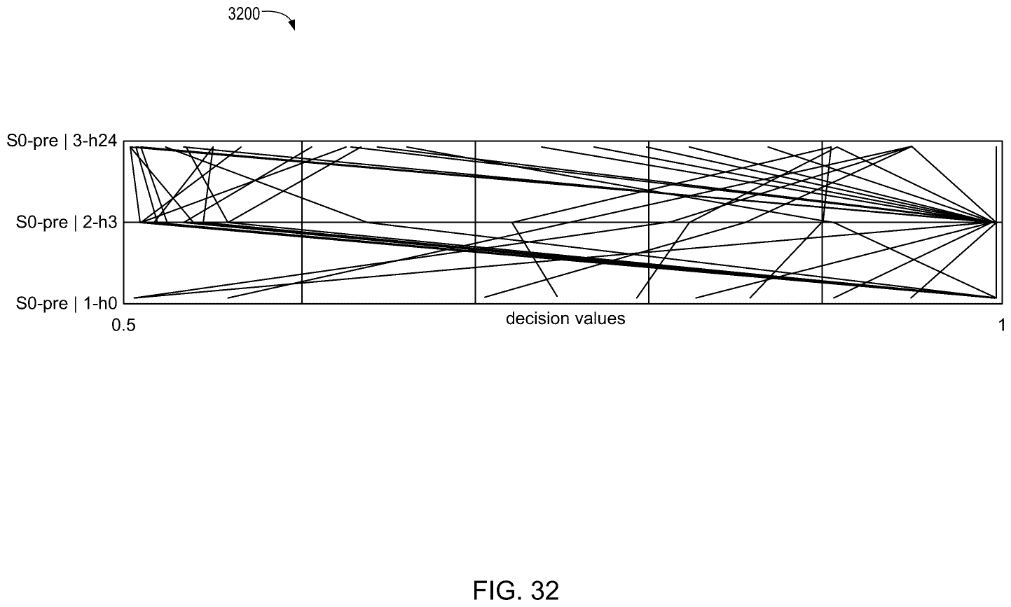

FIG. 32 illustrates a decision value display 3200, according to various embodiments. The illustrated example includes four outcomes and three decisions using compare-to-base as the mechanism for creating decisions. According to various embodiments, the system uses a decision value display to illustrate how features change in importance across binary decisions within the ordered series. The decisions can be created using, for example, the compare-to-base or compare-to-previous techniques described herein, or another decision technique. Each decision is represented as a category on the category axis (vertical axis). The decision values are represented on the value axis (horizontal axis). For each feature F and decision D, the value is the decision value computed for F using the component outcomes of decision D. The category labels are a composite of the labels for outcomes that compose that decision.

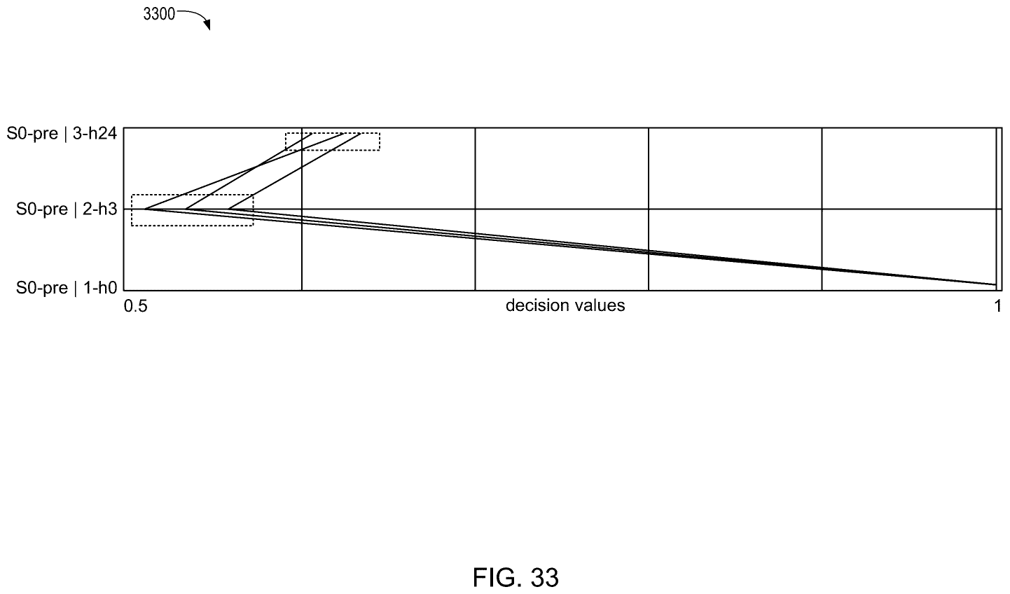

FIG. 33 illustrates the same decision value display 3300 with two selection rectangles, according to one embodiment. The system updates the graphical user interface to show a set of features that are very valuable for one decision and less valuable for later decisions.

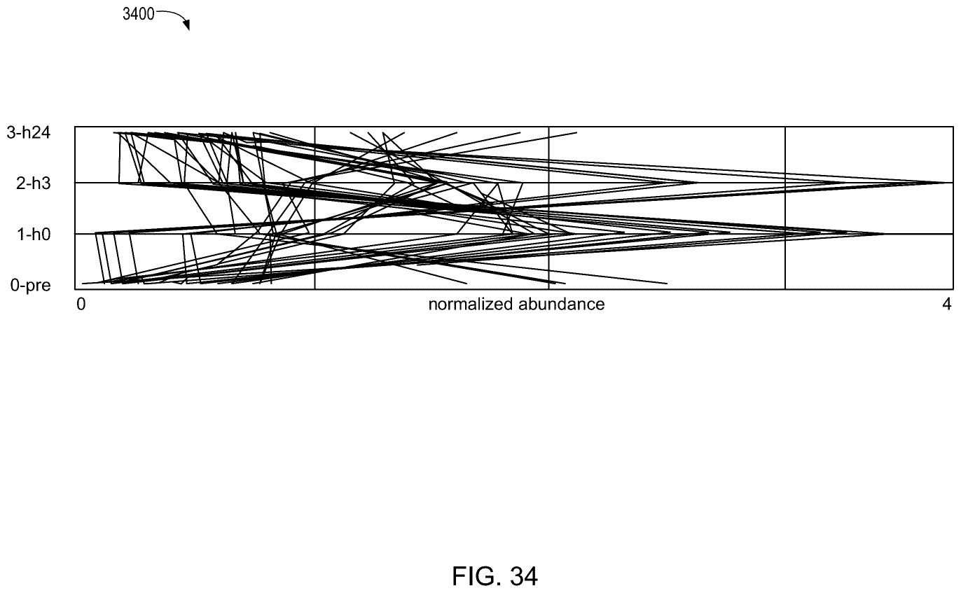

FIG. 34 illustrates an example of an abundance value display 3400 with four outcomes ordered from bottom to top, according to one embodiment. The abundance value display 3400 shows how the abundance of various features will change across the outcome series. In this display, the ordered series of outcomes become the category axis of the display. The value axis is composed of the sum or average of abundance values for a particular feature and outcome.

One problem with using the sum or average is that the abundance values may vary wildly between features, making comparison difficult. Various embodiments of the systems and methods described herein resolve this for a given feature F by computing the average values for each outcome. This provides a value J for each feature decision combination. For a given feature F, the system computes the average of the values for all outcomes to create the feature abundance average, K. The system divides each feature/outcome average J by the respective feature abundance average K. This provides a normalized value for each feature/outcome. J/K values below, equal, to or greater than 1 represent lower, typical, or higher abundances at each outcome, respectively. The system may plot these values by outcome as a line for each feature in the parallel axes display. The result is a representation of how the abundance of a feature varies across an ordered series of outcomes.

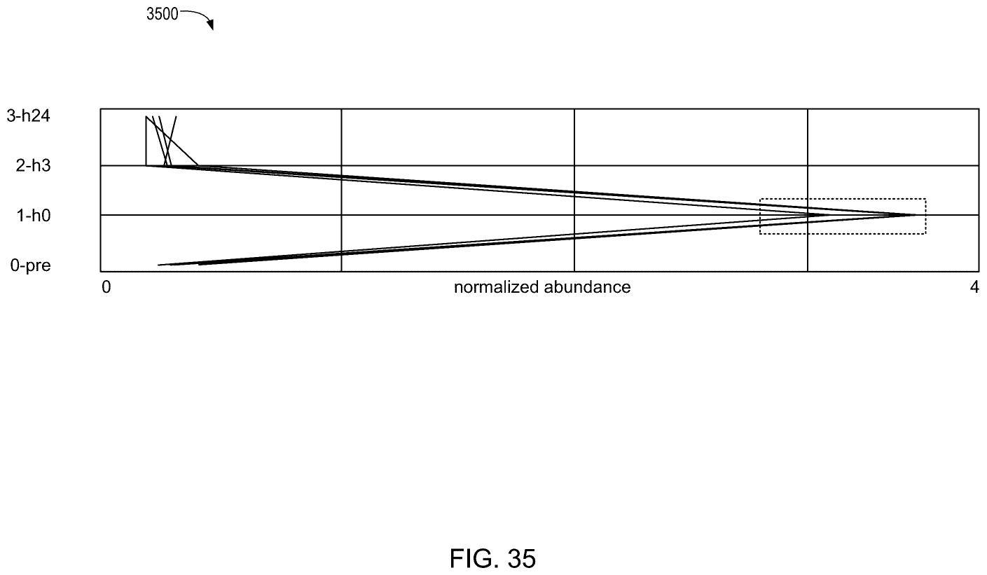

FIG. 35 illustrates the same abundance value display 3500 with a single selection rectangle that selects features with a high abundance in the “1-h0” category. In this example, these all seem to spike for the “1-h0” outcome and then tail off sharply in later categories.

The parallel axes plots, graphs, and displays described herein allow an analyst to easily select, via a graphical user interface, a small set of features of interest. A feature can be represented by a description of that feature. A mass-spectrometry quantum feature can be represented by its bounds in m/z and RT. A K-mer feature can be represented by its nucleotide sequence. Composite features that merge several different quantum and K-mer features can be represented by a list of the component feature descriptions. The system may augment the basic description of a feature with data from an external data source. For example, K-mer features can be used to look up entries in BLAST and information about those entries can be included in the description. It is also possible that MS-2 or other mass-spectrometry sources can produce data that identifies a particular molecule by M/Z and RT. This data can be used to augment the description of a quantum feature.

All or a combination of these information sources can be used to produce a human readable description of a feature. Using the selection techniques described for parallel axes, the system can narrow down the set of selected features. Using this small set of selected features, the system can generate a list of descriptions that will inform the analyst about those features that display particular trends in the decision value or abundance value displays.

Multiple Data Features Derived from Disparate Sources

The system may use mass spectrometry alone or in combination with other techniques described herein to generate extensive data on a wide range of molecule types. When drawing samples from different tissues within the same organism, the system may detect variations in molecular abundances. To prepare molecules for mass spectrometry, the system applies sample preparation techniques. It may subject whole molecules or molecular complexes to mass spectrometry. In some cases, large molecules pose technical challenges. For example, in proteomics, many intact proteins are too large for direct mass spectrometry analysis, so the system fragments them into peptide segments. Different preparation methods may generate peptide profiles with varying abundances. The system may use different chromatography columns to separate molecules in distinct ways.

When analyzing RNA sequencing data, the system processes a sample to generate a set of RNA sequences called runs. Each run consists of sequences ranging from 50 to a few hundred nucleotides, though some may be shorter or longer. To analyze these sequences, the system fragments each run into K-mers, which are unique nucleotide sequences of length K. The system examines all RNA-seq runs using an integer value K, and counts the occurrences of each K-mer. Each K-mer represents a unique data feature, and its frequency indicates the abundance of RNA sequences that contain it, reflecting the organism's RNA expression profile.

Using various tissue sample preparation techniques, the system generates a data vector containing several hundred thousand to several million data points from mass spectrometry and K-mer profiling-based RNA expression. This data vector provides extensive biochemical information about the organism's state.

As previously discussed, the system narrows the feature set by comparing data vectors from two or more subject organism sets. It computes a decision value for each feature to measure its ability to differentiate between the comparison sets. The system then selects the features with the highest decision values as the most relevant for characterizing differences between the sets.

In many cases, data vectors containing millions of points can be reduced to a more manageable number, typically between a few dozen and a few thousand salient data points. The system performs this reduction to streamline analysis while preserving meaningful biological information. An additional subsystem or a separate analytical process may further interpret these data points to support future research on differences between the comparison sets. The condensed data vectors may integrate information from K-mers and multiple mass spectrometry processes. The system applies the methods described herein to derive biological insights from these heterogeneous data sources.

As an example, a protein P may include several hundred amino acids. The system may break down the protein into many peptides that can be detected through mass spectrometry. If the protein P is key or salient to the difference between two sets of subject organisms, then P's component peptides are likely to be similarly expressed. The role of P in the differences between two sets of organisms can be understood in the context of the identification of similarly expressed peptides. P was created from an RNA sequence expressing a particular gene. The K-mers from that RNA sequence should have expression related to P's abundance and the measured abundance of peptides derived from P. The system may use the relationships between K-mer features derived from RNA-seq and peptides identified through proteomic mass spectrometry to understand the underlying biology of differences discovered.

Similarly, the protein P may be relevant to the manufacturing of metabolite M or possibly lipid L or other useful biomolecules. The system can distinguish between and/or otherwise identify these (M or L) using different mass spectrometry techniques. If these are present in the set of data features, then an understanding of their relationship is desirable. Anti-expression may also be informative in some embodiments. Molecule N may suppress the ability for protein P to manufacture lipid L. High expression of N might produce lowered expression of L and vice versa. Molecules N and L are participating together in the same biochemical system, but they are anti-expressed.

For each data feature, whether derived from a K-mer or mass spectrometry data point, the system identifies what the corresponding molecule actually is and how it relates to the metabolic pathways in an organism. The system may, for example, utilize any of a wide variety or combination of existing databases that map molecular measurements to biochemical identities. For example, for nucleotide and amino acid sequences there are the BLAST databases managed by the National Institute of health. As another example, the Klegg database can be used for metabolic pathways and systems. The system may use these databases to look up molecular data for more information.

When working on a particular biological problem there may be a few to a few hundred data points that are identified as important. Each of these may find zero to ten or more matches in the various databases. Each of these matches yields extensive information about the molecule. With 100 features with 10 matches each there are 1,000 multipage documents to assimilate and make sense of. The system may condense this information so that it can be understood.

The system may utilize clustering techniques to address the problem of co-expression can be addressed by clustering techniques. Given that each example can be characterized by a vector of data points, as described above, the system can identify those examples whose data point expression patterns are most similar to each other by clustering those vectors. Given also that each data point has an abundance value for each example, the system can represent each data point by a vector of its example values. They system may cluster data point vectors to visualize co-expression patterns. For example, the system may group together data points whose abundance values exhibit most similar patterns of expression across the same examples. As such, the system may utilize clustering as a basic tool for identifying co-expression patterns for data points.

The system may perform a clustering analysis by taking all data points collected for each example organism in the analysis. As described herein, there may be several million data points per example and they may be from heterogeneous sources, such as K-mers and various methods of mass spectrometry and chromatography. The system may divide the examples into two or more sets that represent different biological conditions. A simplified example is provided below that includes two comparison sets. However, it is appreciated that the techniques described herein can be extended to any number of comparison sets.

The system may compute a decision value for each data point in the comparison sets. The system may compute each decision value according to any of the various embodiments described herein. A given decision value may represent an estimate of how well a particular data point can separate a specific data point's abundance values based on whether they belong to one of the comparison sets versus belonging to the others. In many instances, this results in a reduction in the number of data points for consideration from several million to a few hundred.

According to various embodiments, the system may build an expression matrix using the reduced the number of data points under consideration (e.g., tens or hundreds of data points). The expression matrix may include rows of the data points, and columns of examples. The values in the matrix are the abundance values for each data point for each corresponding example. An example identified as E with a data point D whose value is V may be represented as EM[D,E]=V, where EM is the expression matrix. Throughout this application, rows are used for the data points and columns are used for the examples. It is appreciated that these can be reversed without changing the functionality. Throughout this disclosure, any claim or discussion of rows and columns can be reversed.

Given such an expression matrix, the rows of the matrix are data point vectors that the system can cluster (as described above) to identify co-expression of data points. The columns of the expression matrix are example vectors that the system can cluster (as described above) to identify similar examples. In some embodiments, the system may normalize the rows (data point vectors) prior to clustering by rows. Each data point has a different range of values depending on the frequency of the molecule and the form of analysis used. Normalizing these ranges facilitates a comparison of these data points. There are a variety of normalization techniques, any of which may be utilized. Euclidean normalization divides the vector by its length making all normalized data point vectors of length 1. For example, if the system computes a mean M and standard deviation S for a given data vector, the system can compute a new value V′=(V−M)/S. This centers the mean at zero and normalizes the standard deviation of all vectors to 1. This is known as a z-score. Other normalization schemes may be utilized by the system to normalize the data point vectors (rows) to the same value ranges.

In various embodiments, the system may not normalize the example vectors (columns). Some examples have high values because the molecules being considered are highly expressed in those examples, while other examples are not expressed on those molecules. Normalization of example columns might destroy those salient differences. In embodiments in which the system implements anti-expression as well as co-expression, the system may take the absolute value of the normalized values. This makes all values positive so that down-expressed data points can be directly compared to up-expressed data points. This will capture anti-expression relationships.

Many clustering algorithms are based on a distance metric that compares two vectors. The Euclidean metric computes the Euclidean distance between the vectors. The cosine metric computes the cosign between two unit-vectors (length 1) as their dot product. The Manhattan distance sums the absolute values of the differences in each vector element. Other distance metrics are possible. Not all features are equally important. The decision value for each feature is a measure of such importance. When comparing examples, each example has a vector of data points. As part of the distance metric for measuring distances between examples, the system may multiply each data point by that feature's importance before applying the distance metric to emphasize differences between important data points and deemphasize distances between less important data points.

There are a variety of algorithms for clustering objects that have a distance metric defined between them. Agglomerative clustering starts with each object being a cluster. In embodiments utilizing agglomerative clustering, the two neighboring clusters nearest to each other are combined into a larger cluster. The two neighboring clusters are removed and replaced by the new combined cluster. This process is repeated until there is only one cluster that contains all the objects to be clustered. K-means clustering begins by dividing a single cluster of all objects into two clusters each represented by the mean of the objects in that cluster. Each cluster is then similarly divided until each leaf cluster contains only one object. There are other clustering algorithms and variants on these algorithms, any of which may be utilized by the system.

Clustering algorithms can be used to create a tree of similar objects in each cluster. In various embodiments, the tree is displayed to allow a user to interactively select any node of that tree (e.g., via a graphical user interface or GUI). Selection of a cluster tree node selects all the objects in that cluster, creating an embedded cluster tree with the selected node the main trunk of the resulting tree. The system may then summarize (e.g., via a GUI for the user) the information within that selected set of objects. For example, selecting a tree node from clustering examples, a subset of examples is specified. As an example, a summary of the examples may include a count of each of the various outcomes or other example properties associated with those examples. The rendered visualization may facilitate an understanding of how well the outcomes correlate with similarly clustered examples.

### Bi-Cluster Interaction

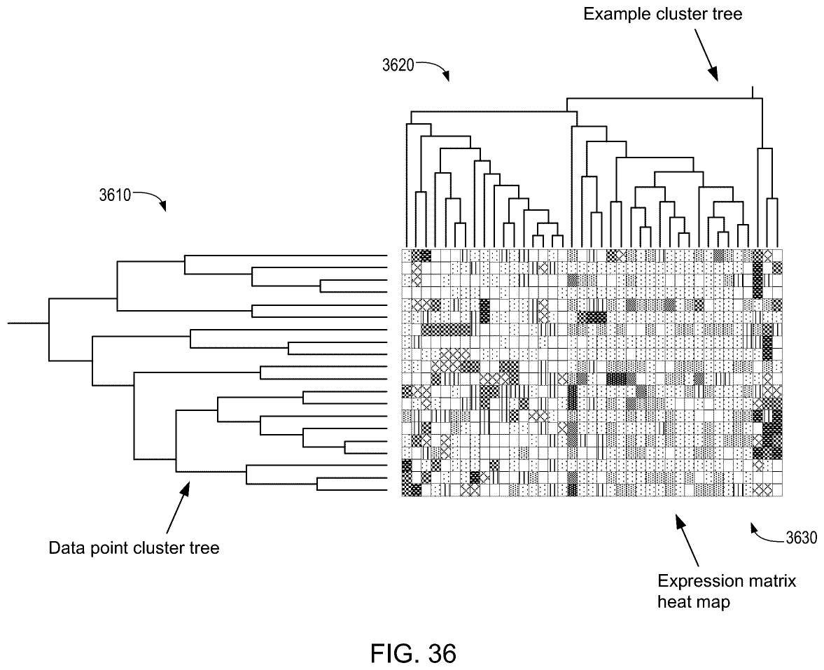

FIG. 36 illustrates a bi-cluster display that enables interaction with examples and data points selected to characterize those examples. This bi-cluster display includes an expression matrix heat map 3630, an example cluster tree 3620, and a data point cluster tree 3610. The expression matrix heat map 3630 represents normalized values from the expression matrix. The system may encode values using various colors or in grayscale. In the grayscale variant illustrated, white represents values near zero, darker shades indicate increasing positive values, and lighter shades correspond to negative values. In a color representation, values at or near zero are given one color (e.g., white), the largest positive value is given another color (e.g., green), and positive values are a continuous blend between that largest positive color and the zero color. Similarly, the smallest negative value may be assigned a third color (e.g., red) and negative values of the expression matrix may be displayed as a continuous blend between the zero color and the smallest negative color.

The visual representation allows users to quickly discern relationships between values in the matrix. In FIG. 36, three distinct groups of examples emerge, demonstrating clear clustering patterns among the dataset. Additional information may be used to analyze why these groups differ. Notably, even within closely clustered pairs, data points in the expression matrix heat map 3630 do not exhibit strong similarity, indicating a lack of co-expression among features. The data point cluster tree 3610 arranges similar data points adjacent to each other along the Y-axis. The example cluster tree 3620 organizes similar examples along the X-axis, ensuring that adjacent examples share higher similarity.

Aligning the expression matrix heat map 3630 with the two cluster trees 3610 and 3620 enables users to identify patterns among both examples and data points. The placement of axes is flexible, such that data points and examples can be swapped along the X and Y axes without affecting functionality. Additionally, the example cluster tree 3620 could be moved to the bottom, and the data point cluster tree 3610 could be placed on the right, without altering the interpretation of the visualization.

### Interactivity

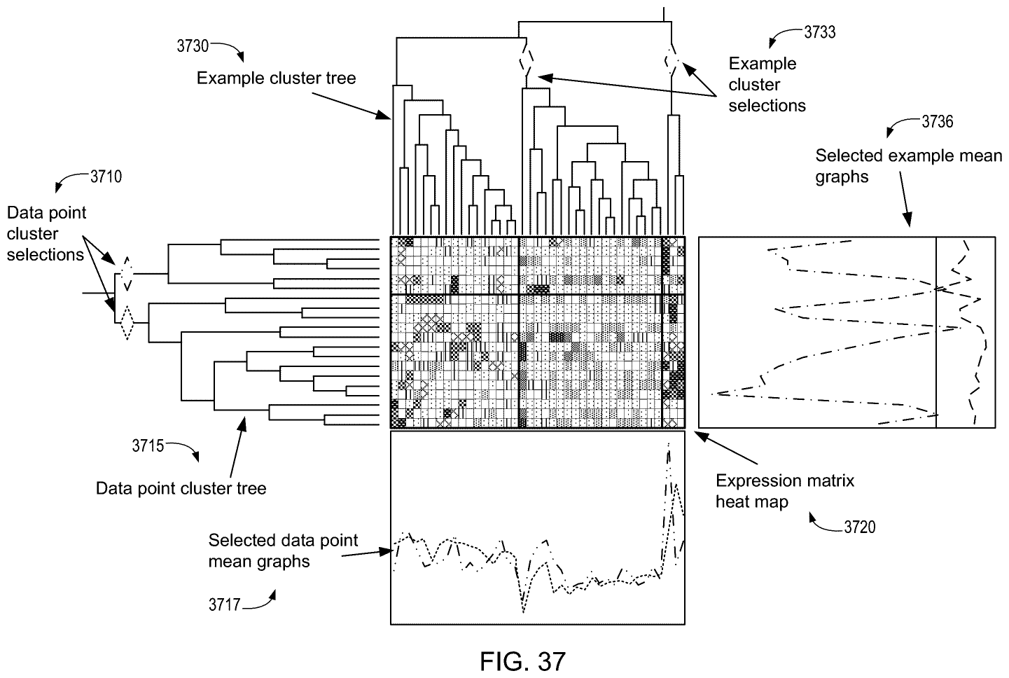

FIG. 37 illustrates two example cluster selections and two data point cluster selections. According to various embodiments, the system may generate a graphical user interface (GUI) that allows a user to interact with this display by selecting one or more nodes of the cluster trees. In a color rendition, each cluster selection may represented by a colored diamond. Any other distinct shape could be used, as could altering the properties (color, thickness, pattern, etc.) of the cluster tree lines. In a color embodiment, a colored shape at the node may be used to identify selected clusters. Different cluster selections may be distinct or nested. Different shapes could also be used to identify the clusters. Cluster nodes can be selected by clicking on them with a mouse, pen or using a touch screen. Clicking on an already selected node can unselect it.

FIG. 37 shows the selection 3733 of two nodes in the example cluster tree 3730. Because examples are represented as columns of values, the system can compute the mean column vectors for each example cluster, producing a new mean vector for each example cluster selection. These mean vectors are plotted in the selected example mean graphs 3736 on the right side of the figure. In a color version, each uses the color of its cluster's selection mark. In the grayscale version, different line patterns are utilized. From the selected example mean graphs 3736, a user can readily see how very different these two example clusters are from each other.

In a similar fashion, the illustrated example includes two data point cluster selections 3710 on the left side. Because data points are represented as row vectors in the expression matrix 3720, the system can compute a mean vector for each selected data point cluster and plot those vectors in the selected data point mean graphs 3717, as shown at the bottom of the figure. In the data point mean graphs 3717, the mean vectors are different, but not significantly so. They are not as clearly distinct as the selected example mean graphs 3736. This indicates that there is not a lot of co-expression among the data points. FIG. 37 is an example graphical display of the normalized values of the mean vectors. This tends to capture the shape of the differences between the means. The graphs could also use the non-normalized values from the expression matrix 3720 to characterize the data more accurately.

A user may also desire to understand what the cluster groups mean biologically. For each object in the cluster tree 3730, the system can add additional information known about that object. For example, the system might add gender, weight, age, racial origin, species, body mass index, etc. to example objects. For data points in the data point cluster tree 3715, the system might add chemical formula, chemical name, organs where this molecule functions, etc. Having selected a particular cluster group, the user may want a summary of the information found with all member objects in the group. For each object, the system provides a set of zero or more named attributes and then summarize each of those attributes to produce a profile of the cluster as a whole.

The system may deal with various types of information about objects in a cluster tree, including numeric information and words. Numeric information can be summarized in different ways, including using a mean, mean plus standard deviation, median (50th percentile), or a median plus the 20th and 80th percentile. Other numeric summaries are possible. These might be applied to numeric attributes like weight, age, or body mass index.

One approach to summarize word attributes is to use simple category attributes, where the attribute consists of one of a small number of possible categories. In the example data shown in FIG. 37, each example is for early-stage or late-stage breast cancer. For a given cluster group, the system can count how many are early and how many are late and show those counts. The right-most selected group of examples are all late-stage patients. The middle-selected group is mostly early-stage patients with one late-stage patient. This kind of summary quickly captures part of the biological meaning of the groups but also points out a question for further exploration (e.g., why is the one late-stage patient grouped with all the early-stage patients?).

In some instances, word-based information is given in a semi-structured form. Various names and categories are given in no particular order. This information can be hard to summarize. A given object/attribute value may simply be a sequence of words. Human readers make perfect sense of them, but the automated summary may not. One way to summarize such an attribute is to count how many times each word appears in the group and then sort them from highest to lowest count. This sorted word list quickly gives meaning to the group.

With sorted word count lists, the system may get high-frequency words that have little meaning. In biology it is common to provide an organism classification from phylum down to species. If the examples are all primates, then all categories for mammals and above have high counts but little helpful information. For a given word, the system can divide its count by the number of objects in the entire tree that have that word associated with it. Very common words will get lower scores and the words more unique to the group will get higher scores because they have smaller denominators. Words that occur infrequently in the group will also get lower scores because they will have lower numerator counts.

This disclosure has been made with reference to various exemplary embodiments, including the best mode. However, those skilled in the art will recognize that changes and modifications may be made to the exemplary embodiments without departing from the scope of the present disclosure. While the principles of this disclosure have been shown in various embodiments, many modifications of structure, arrangements, proportions, elements, materials, and components may be adapted for a specific environment and/or operating requirements without departing from the principles and scope of this disclosure. These and other changes or modifications are intended to be included within the scope of the present disclosure.

This disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element. This disclosure should, therefore, be understood to encompass at least the following claims and all possible permutations thereof.

1. A method for identifying salient biological features from molecular data to differentiate between biological outcomes, the method comprising:
obtaining biological samples from a plurality of subjects;
preparing each biological sample to form a sample analyte;
analyzing each sample analyte to generate molecular feature data for each respective biological sample;
generating a set of feature vectors from the molecular feature data of the biological samples, wherein each feature vector comprises numerical values indicative of molecular abundance within each respective biological sample;
forming a training set of feature vectors by associating each feature vector with a known biological outcome from at least two outcome categories;
computing, for each distinct feature across the training set, a decision value that indicates an ability of that feature to distinguish among the outcome categories;
selecting a pruned, salient feature set by discarding features having decision values below a reliability threshold;
constructing a classification model using the pruned, salient feature set, the classification model comprising parameters to distinguish among the outcome categories; and
applying the classification model to a new biological sample by:
analyzing the new biological sample to generate a new feature vector,
extracting features from the new feature vector that correspond to the pruned, salient feature set, and
generating a predicted biological outcome for the new biological sample based on the classification model.

2. The method of claim 1, wherein preparing the biological sample to form the sample analyte comprises at least one of: removing cells or coagulated components to form a plasma or serum, applying a chemical treatment, applying a mechanical treatment, applying an enzymatic treatment, fragmenting proteins in the biological sample, and extracting nucleic acids from the biological sample.

3. The method of claim 1, wherein analyzing the sample analyte to generate molecular feature data comprises using a mass spectrometer to perform a mass spectrometry analysis.

4. The method of claim 3, wherein the mass spectrometry analysis comprises:
passing the sample analyte through a chromatography column to separate molecular components according to differential retention times;
ionizing the molecular components of the sample analyte,
detecting abundance signals for a range of mass-to-charge values (M/Z) over a series of the retention times, and
converting the abundance signals from the mass spectrometer into feature vectors representing molecular abundance.

5. The method of claim 1, wherein analyzing the sample analyte to generate molecular feature data comprises using a sequencing device to generate nucleotide sequence reads.

6. The method of claim 5, wherein analyzing the sample analyte to via the sequencing device comprises:
generating short-read sequences of nucleotides,
converting the short-read sequences into K-mer features, and
forming each feature vector based on abundances of K-mer features.

7. The method of claim 1, wherein computing the decision value for each feature of each respective feature vector, comprises:
partitioning measured feature values into two or more intervals, and
quantifying how distinctly each interval separates the outcome categories by computing at least one purity or impurity measure,
wherein higher decision values correspond to improved separation of the outcome categories.

8. The method of claim 7, wherein quantifying how distinctly each interval separates the outcome categories comprises using at least one statistical approach selected from a group of statistical approaches consisting of: a GINI purity separation test, a parametric statistical test, and an equiprobable distribution test.

9. The method of claim 1, wherein selecting a pruned, salient feature set comprises evaluating the quality of a feature by determining the reliability threshold based on a random decision value for a randomly shuffled set of outcomes.

10. The method of claim 1, wherein constructing the classification model comprises:
training a machine-learning classifier with the pruned, salient feature set and the known outcomes, the classifier comprising at least one of a decision tree and a neural network;
optimizing model parameters of the machine-learning classifier to reduce misclassification rates via cross-validation; and
storing the trained model parameters in a non-transitory computer-readable medium for subsequent outcome predictions.

11. The method of claim 1, wherein applying the classification model to the new biological sample further comprises generating a confidence score indicative of how distinctly the pruned, salient feature set classifies the new biological sample into one of the outcome categories.

12. A system to identify salient biological features from molecular data to differentiate between biological outcomes, comprising:
a sample preparation module configured to receive a plurality of biological samples from respective subjects and to prepare each biological sample to form a sample analyte;
a molecular analysis device configured to analyze each sample analyte and generate molecular feature data for each respective biological sample, wherein the molecular feature data are indicative of molecular abundance; and
a computing device comprising at least one processor at a non-transitory computer-readable medium storing instructions which, when executed by the at least one processor, cause the computing device to:
generate a set of feature vectors from the molecular feature data of the biological samples, each feature vector comprising numerical values indicative of molecular abundance within each respective biological sample;
form a training set of feature vectors by associating each feature vector with a known biological outcome from at least two outcome categories;
compute, for each distinct feature across the training set, a decision value that indicates an ability of that feature to distinguish among the outcome categories;
select a pruned, salient feature set by discarding features having decision values below a reliability threshold;
construct a classification model using the pruned, salient feature set, the classification model comprising parameters to distinguish among the outcome categories; and
apply the classification model to a new biological sample by:
analyzing the new biological sample to generate a new feature vector,
extracting features from the new feature vector that correspond to the pruned, salient feature set, and
generating a predicted biological outcome for the new biological sample based on the classification model.

13. The system of claim 12, wherein the sample preparation module is further configured to perform at least one of: removing cells or coagulated components to form a plasma or serum, applying a chemical treatment, applying a mechanical treatment, applying an enzymatic treatment, fragmenting proteins in the biological sample, and extracting nucleic acids from the biological sample.

14. The system of claim 12, wherein the molecular analysis device comprises a mass spectrometer configured to perform a mass spectrometry analysis on the sample analyte to generate the molecular feature data.

15. The system of claim 14, wherein the mass spectrometer is further configured to:
pass the sample analyte through a chromatography column to separate molecular components according to differential retention times;
ionize the molecular components of the sample analyte;
detect abundance signals for a range of mass-to-charge (m/z) values over a series of retention times; and
convert the abundance signals into feature vectors representing molecular abundance.

16. The system of claim 12, wherein the molecular analysis device comprises a sequencing device configured to generate nucleotide sequence reads as the molecular feature data for each sample analyte.

17. The system of claim 16, wherein the sequencing device is further configured to:
generate short-read sequences of nucleotides;
convert the short-read sequences into K-mer features; and
form each feature vector based on abundances of K-mer features.

18. The system of claim 12, wherein the computing device, in computing the decision value for each distinct feature, is configured to:
partition measured feature values into two or more intervals;
quantify how distinctly each interval separates the outcome categories by computing at least one purity or impurity measure; and
assign higher decision values to features exhibiting improved separation of the outcome categories.

19. The system of claim 18, wherein the computing device is configured to implement at least one statistical approach for quantifying how distinctly each interval separates the outcome categories, the at least one statistical approach selected from the group consisting of: a GINI purity separation test, a parametric statistical test, and an equiprobable distribution test.

20. The system of claim 12, wherein, to select the pruned, salient feature set, the computing device is configured to evaluate features by determining the reliability threshold based on a random decision value generated by a randomly shuffled set of outcomes.

21. The system of claim 12, wherein the computing device is further configured to construct the classification model by:
training a machine-learning classifier with the pruned, salient feature set and the known outcomes, the classifier comprising at least one of a decision tree or a neural network,
optimizing model parameters to reduce misclassification rates via cross-validation, and
storing the trained model parameters in the non-transitory computer-readable medium for subsequent outcome predictions.

22. The system of claim 12, wherein, when applying the classification model to the new biological sample, the computing device is further configured to generate a confidence score indicative of how distinctly the pruned, salient feature set classifies the new biological sample into one of the outcome categories.

23. A method of determining a predicted biological outcome for a biological sample using a classification model, the method comprising:
obtaining a classification model configured to distinguish among at least two biological outcome categories, wherein the classification model is based on a pruned, salient feature set associated with known biological outcomes;
receiving a biological sample from a subject;
analyzing the biological sample to generate one or more feature vectors, each comprising numerical values indicative of molecular abundance in the biological sample;
extracting, from the one or more feature vectors, those features identified by the classification model as part of the pruned, salient feature set; and
applying the classification model to the extracted features to produce a predicted biological outcome for the biological sample and providing an output indicative of the predicted biological outcome.

24. The method of claim 23, wherein the classification model was previously constructed by:
receiving a training set of feature vectors associated with known biological outcomes;
computing, for each distinct feature across the training set, a decision value indicating an ability of that feature to differentiate among the outcome categories;
generating the pruned, salient feature set by discarding features having decision values below a reliability threshold; and
generating the classification model based on the pruned, salient feature set.
